(19) United States
(12) Patent Application Publication
(10) Pub. No.: US 2012/0196858 A1
(43) Pub. Date: Aug. 2, 2012

(54) METHOD OF PROMOTING APOPTOSIS AND INHIBITING METASTASIS

(75) Inventor: David Schlaepfer, San Diego, CA (US)

(73) Assignee: Poniard Pharmaceuticals ,Inc., Seattle, WA (US)

(21) Appl. No.: 13/389,417

(22) PCT Filed: Aug. 12, 2010
(86) PCT No.: PCT/US2010/045359

§ 371 Date: Apr. 20, 2012

Related U.S. Application Data
(60) Provisional application No. 61/233,351, filed on Aug. 12, 2009.

(51) Int. Cl. A61K 31/5377; A61P 35/00; A61P 35/04; C07D 413/12

(52) U.S. Cl. 514/235.5; 544/131

(57) ABSTRACT

The invention provides a method of promoting apoptosis in tumor cells, which can result in inhibiting tumor growth, or inhibiting tumor metastasis, or promoting tumor apoptosis, or any combination thereof, by administration of an effective amount of a focal adhesion kinase (FAK) inhibitor. The inhibitor is a small molecule organic compound. Accordingly, the focal adhesion kinase inhibitor can be used in the treatment of tumors, such as malignant cancer. For example, administration of effective amounts of the FAK inhibitor PND-1186 has been found to inhibit tumor cells in murine models for breast and ovarian cancer.

### CROSS-REFERENCE TO RELATED APPLICATION

This application claims the priority of U.S. Ser. No. 61/233,351, filed Aug. 12, 2009, which is incorporated by reference herein in its entirety.

### STATEMENT OF GOVERNMENT SUPPORT

This invention was made with government support under NIH grants CA107263 and CA102310 made by the National Institutes of Health, and by a US Army Medical Research grant OC080051. The U.S. government has certain rights in the invention.

### BACKGROUND

Apoptosis is a cellular phenomenon wherein cells undergo a programmed death in response to internal biochemical signals. Typically, apoptosis results in advantages to the organism, being involved in development and differentiation. It is believed that in some cases tumors proliferate due to a failure to undergo apoptosis. Tumor proliferation and metastasis are negative processes for the organism containing the tumor. Tumor metastasis is a leading cause of cancer-related death.

Tumor cells can grow in an anchorage-independent manner. This is mediated in part through survival signals that bypass normal growth restraints controlled by integrin cell surface receptors.

Focal adhesion kinase (FAK) is a cytoplasmic tyrosine kinase recruited to integrin-mediated matrix attachment sites where FAK activity is implicated in the control of cell survival, migration, and invasion. Focal adhesion kinase (FAK) associates with integrins and modulates various cellular processes including growth, survival, and migration.

FAK acts as both a signaling kinase and cell adhesion-associated scaffold within tumor cells to coordinate the positional recruitment and phosphorylation of various cytoskeletal-associated proteins such as p130Cas and paxillin. See Schlaepfer D D, Hauck C R, Sieg D J., Signaling through focal adhesion kinase, Prog Biophys Mol Biol 1999, 71:435-78; Zouq N K, Keeble J A, Lindsay J, Valentijn A J, Zhang L, Mills D, et al., FAK engages multiple pathways to maintain survival of fibroblasts and epithelia: differential roles for paxillin and p130Cas, J Cell Sci 2009, 122:357-67. Increased FAK autophosphorylation at Y397 is a marker of FAK activation. Integrin-mediated Y397 FAK phosphorylation can promote Src-family tyrosine kinase binding to FAK and can lead to FAK-mediated c-Src activation. See Wu L, Bernard-Trifilo J A, Lim Y, Lim S T, Mitra S K, Uryu S, et al., Distinct FAK-Src activation events promote alpha5beta1 and alpha4beta1 integrin-stimulated neuroblastoma cell motility, Oncogene 2008, 27:1439-48. As both FAK and c-Src can phosphorylate common downstream targets such as p130Cas, it remains undetermined whether the effects of FAK and/or c-Src inhibition will yield differential results on downstream target phosphorylation events. See Defilippi P, Di Stefano P, Cabodi S. p130Cas: a versatile scaffold in signaling networks, Trends Cell Biol 2006, 16:257-63. In murine 4T1 breast carcinoma cells, it has been shown that FAK promotes an invasive and metastatic cell phenotype. See Mitra S K, Lim S T, Chi A, Schlaepfer D D, Intrinsic focal adhesion kinase activity controls orthotopic breast carcinoma metastasis via the regulation of urokinase plasminogen activator expression in a syngeneic tumor model, Oncogene 2006, 25:4429-40. FAK expression is elevated in invasive humans cancers (Mitra S K, Schlaepfer D D., Integrin-regulated FAK-Src signaling in normal and cancer cells, Curr Opin Cell Biol 2006, 18:516-23) and FAK signaling promotes directional cell movement (Mitra S K, Hanson D A, Schlaepfer D D., Focal adhesion kinase: in command and control of cell motility, Nat Rev Mol Cell Biol 2005, 6:56-68; Tomar A, Schlaepfer D D., Focal adhesion kinase: switching between GAPs and GEFs in the regulation of cell motility, Curr Opin Cell Biol 2009, 21:676-83).

ATP-competitive small molecule inhibitors to FAK have been developed by Novartis (TAE-226) and Pfizer (PF-573,228, PF-562,271). Additionally, compounds (such as Y15) have been identified that block access to the major FAK tyrosine-397 autophosphorylation and Src-family kinase binding to FAK. See, for example, Shi Q, Hjelmeland A B, Keir S T, Song L, Wickman S, Jackson D, et al., A novel low-molecular weight inhibitor of focal adhesion kinase, TAE226, inhibits glioma growth, Mol Carcinog 2007, 46:488-96; Slack-Davis J K, Martin K H, Tilghman R W, Iwanicki M, Ung E J, Autry C, et al., Cellular characterization of a novel focal adhesion kinase inhibitor, J Biol Chem 2007, 282:14845-52; Roberts W G, Ung E, Whalen P, Cooper B, Hulford C, Autry C, et al., Antitumor activity and pharmacology of a selective focal adhesion kinase inhibitor, PF-562,271, Cancer Res 2008, 68:1935-44; and Golubovskaya V M, Nyberg C, Zheng M, Kweh F, Magis A, Ostrov D, et al., A small molecule inhibitor, 1,2,4,5-benzenetetraamine tetrahydrochloride, targeting the y397 site of focal adhesion kinase decreases tumor growth, J Med Chem 2008, 51:7405-16.

Various inhibitors of FAK, including PND-1186, are disclosed in PCT patent application number PCT/US2008/003205, filed Mar. 10, 2008, and published as WO 2008/115369, which is incorporated by reference herein in its entirety. The preparation of PND-1186 is described in that published patent application.

### SUMMARY

The present invention is directed to a method of promoting cellular apoptosis, or inhibiting metastasis in a patient, or both, in vivo, such as in a patient, comprising administering an effective amount of a small molecule inhibitor of focal adhesion kinase to a patient in need thereof. For example, the inhibitor can be a compound of formula

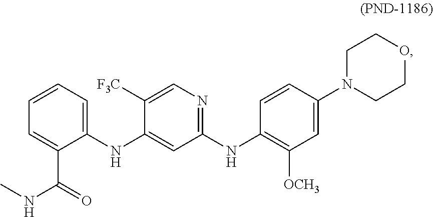

or any pharmaceutically acceptable salt thereof.

### BRIEF DESCRIPTION OF THE FIGURES

FIG. 1 shows K-LISA (Calbiochem) activity profile of GST-FAK 411-686 and His-tagged FAK 411-686 (Millipore) measuring the phosphorylation of poly Glu:Tyr (4:1). After baculovirus expression, glutathione agarose binding, and size fractionation chromatography, the purity of GST-FAK 411-686 was >90% as visualized by SDS-PAGE and Coomassie Blue staining (shown). Average values±SD were determined by triplicate analysis.

FIG. 2 shows a series of immunoblotted SDS-PAGE bands for the indicated proteins, showing the effect of PND-1186 on FAK, c-Src and p130Cas tyrosine phosphylation. Increased FAK autophosphorylation at Y397 is a marker of FAK activation. 4T1 cells were seeded at 70% confluency on tissue culture plates coated with 10 μg/mL fibronectin. Cells were treated with vehicle (DMSO) or with (A) PND-1186 or (B) dasatinib (LC Labs Inc.) for 1 h. Shown is total FAK, p130Cas, Src or actin levels in cell lysates. Phospho-specific immunoblotting was performed in parallel for changes in FAK or Src activity (pY397 FAK or pY416 Src) and p130Cas tyrosine phosphorylation (pY249 p130Cas). (C) Time Course of FAK pY397 phosphorylation in 4T1 cells after 1 h treatment (PND-1186, 1 μM) followed by PBS wash. Protein lysates were made at indicated times after PBS wash.

FIG. 3 shows: (A) a series of time-lapse microscopic images from wound assay at 0, 11, and 22 hr (scale bar is 250 μM) in the presence of vehicle (DMSO) or 1 μM PND-1186; (B) Quantification of time-lapse images from one representative experiment in triplicate; (C) motility of cells on fibronectin-coated MilliCell transwells after 4 h in the presence of 1 μM PND-1186 as percent of DMSO control (±SD).

FIG. 4 shows: (A) adherent and suspended (non-adherent) growth of 4T1 cells on culture plates in the presence of vehicle and indicated concentrations of PND-1186 over 72 hours; (B) cell cycle analyses of adherent and suspended growth of cells using propidium iodide staining, relative DNA content is indicated; (C) stained SDS-PAGE bands showing lysates from adherent or suspended cells showing caspase 3 cleavage; (D) graph of annexin V positive cells determined using flow cytometry of adherent versus suspended cells in the presence of the indicated concentrations of PND-1186.

FIG. 5 shows: (A) photomicrograph of 4T1 cell spheroid suspension in the presence of indicated concentrations of PND-1186; (B) graph of spheroid size at 72 h (n=40)±SD; (C) immunoblotted SDS-PAGE bands showing activity of indicated concentrations of PND-1186 on indicated proteins.

FIGS. 6 (A) and (D) are photomicrographs of 4T1 cell colonies on soft agar in the presence of the indicated concentrations of PND-1186; (B), (C), and (E) are graphs showing the relationship of colony number, colony size, and percent cell apoptosis, respectively, in the presence of the indicated concentrations of PND-1186.

FIG. 7 shows: (A) photomicrographs of subcutaneously implanted mCherry 4T1 tumors after 8 days in mice in the presence of 100 mg/kg PND-1186 versus control; (B) a graph showing tumor weight of the subcutaneously implanted tumors after 8 days in mice in the presence of the indicated quantities of PND-1186; (C) quantification of average fluorescent TUNEL staining intensity versus the indicated quantities of PND-1186; (D) representative photomicrograph images of TUNEL-stained tumors in the presence of the indicated quantities of PND-1186; (E) fluorescent-stained photomicrographs indicating cleaved caspase-3 in cells from implanted tumor exposed to 100 mg/kg PND-1186 compared to control.

FIG. 8 shows: (A) and (B) cell numbers and viable cell numbers, respectively, from ovarian carcinoma tumors ID8 cells plated in the presence of the indicated concentrations of PND-1186; (C) photographs of C57BI6 mice injected intraperitoneally with ID8 cells after 46 days, the test mouse having been provided with 0.5 mg/mL PND-1186 orally ad libitum (in 5% sucrose solution) versus control; (D) a graph of ascites-associated cells collected from the peritoneal cavity of the ID8-injected mice versus controls; (E) immunoblotted SDS-PAGE bands with anti-FAK followed by phospho-specific anti-FAK in mouse treated with 0.5 mg/mL PND-1186 versus control; (E) graph showing ratio of FAK phosphorylation to total FAK in ascites cells from mice; (G) brightfield and fluorescent photomicrograph images of peritoneal tissue from ID8-injected mice after exposure to 0.5 mg/mL PND-1186 versus control; (H) graph showing quantification of peritoneal-associated tumors from ID8-injected mice as above.

### DETAILED DESCRIPTION

As used in the specification and the appended claims, the singular forms “a,” “an” and “the” include plural referents unless the context clearly dictates otherwise.

As used herein, “individual” or “patient” (as in the subject of the treatment) means both mammals and non-mammals. Mammals include, for example, humans; non-human primates, e.g. apes and monkeys; and non-primates, e.g. dogs, cats, cattle, horses, sheep, and goats. Non-mammals include, for example, fish and birds.

The expression “effective amount”, when used to describe therapy to an individual suffering from a disorder, refers to the amount of a compound of the invention that is effective to inhibit or otherwise act on FAK in the individual's tissues wherein FAK involved in the disorder is active, wherein such inhibition or other action occurs to an extent sufficient to produce a beneficial therapeutic effect.

“Treating” or “treatment” within the meaning herein refers to an alleviation of symptoms associated with a disorder or disease, or inhibition of further progression or worsening of those symptoms, or prevention or prophylaxis of the disease or disorder, or curing the disease or disorder.

As used herein, an “effective amount” or a “therapeutically effective amount” of a compound of the invention refers to an amount of the compound that alleviates, in whole or in part, symptoms associated with the disorder or condition, or halts or slows further progression or worsening of those symptoms, or prevents or provides prophylaxis for the disorder or condition. In particular, a “therapeutically effective amount” refers to an amount effective, at dosages and for periods of time necessary, to achieve the desired therapeutic result. A therapeutically effective amount is also one in which any toxic or detrimental effects of compounds of the invention are outweighed by the therapeutically beneficial effects.

“Apoptosis” as the term is used herein refers to a programmed death of a cell or a group of cells, wherein internal biochemical mechanisms come into effect, either from endogenous or exogenous signals, that bring about the eventual death of the cell. “Promoting” apoptosis is an action by an exogenous signal, a FAK kinase inhibitor molecule, that results in apoptosis in a cell that might otherwise continue existence, e.g., in a tumor (i.e., “promoting tumor apoptosis” refers to this process when the action is selective to some degree for cells in a tumor compared to normal cells).

“Inhibiting tumor growth” refers to an effect on size or mass increase of a tumor wherein the increase is diminished relative to what would be expected in the absence of an effective amount of the FAK inhibitor.

“Inhibiting tumor metastasis” refers to an effect of reducing the incidence or rate of metastasis, or malignant transformation, of cells in a population of tumor cells, and inhibiting the induction of a cancerous state in normal cells by migrating cancer cells.

A “salt” as is well known in the art includes an organic compound such as a carboxylic acid, a sulfonic acid, or an amine, in ionic form, in combination with a counterion. For example, acids in their anionic form can form salts with cations such as metal cations, for example sodium, potassium, and the like; with ammonium salts such as NH₄⁺ or the cations of various amines, including tetraalkyl ammonium salts such as tetramethylammonium, or other cations such as trimethylsulfonium, and the like. A “pharmaceutically acceptable” or “pharmacologically acceptable” salt is a salt formed from an ion that has been approved for human consumption and is generally non-toxic, such as a chloride salt or a sodium salt. A “zwitterion” is an internal salt such as can be formed in a molecule that has at least two ionizable groups, one forming an anion and the other a cation, which serve to balance each other. For example, amino acids such as glycine can exist in a zwitterionic form. A “zwitterion” is a salt within the meaning herein. The compounds of the present invention may take the form of salts. The term “salts” embraces addition salts of free acids or free bases which are compounds of the invention. Salts can be “pharmaceutically-acceptable salts.” The term “pharmaceutically-acceptable salt” refers to salts which possess toxicity profiles within a range that affords utility in pharmaceutical applications. Pharmaceutically unacceptable salts may nonetheless possess properties such as high crystallinity, which have utility in the practice of the present invention, such as for example utility in process of synthesis, purification or formulation of compounds of the invention.

Suitable pharmaceutically-acceptable acid addition salts may be prepared from an inorganic acid or from an organic acid. Examples of inorganic acids include hydrochloric, hydrobromic, hydriodic, nitric, carbonic, sulfuric, and phosphoric acids. Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid. Examples of pharmaceutically unacceptable acid addition salts include, for example, perchlorates and tetrafluoroborates.

The present invention is directed to the use of small molecule inhibitors of FAK, such as PND-1186, for promotion of cellular apoptosis, or inhibiting metastasis in a patient, or both, in vivo. The small molecule FAK inhibitors can block FAK Tyr-397 phosphorylation in vivo and can exhibit anti-tumor efficacy, such as in reducing tumor size and preventing tumor metastasis, by inducing or promoting tumor cell apoptosis.

The present invention is directed to a method of promoting cellular apoptosis, or inhibiting metastasis in a patient, or both, such as in a patient, comprising administering an effective amount of an inhibitor of focal adhesion kinase to a patient in need thereof. For example, the inhibitor can be a compound of formula

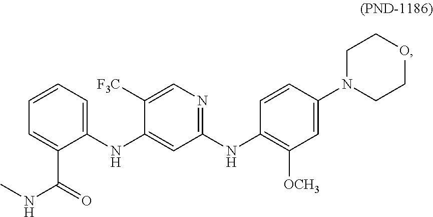

or any pharmaceutically acceptable salt thereof. Various pharmaceutically acceptable salts are discussed below. Other related FAK inhibitors are disclosed in PCT patent application number PCT/US2008/003205, filed Mar. 10, 2008, and published as WO 2008/115369, incorporated herein by reference.

In various embodiments, promoting cellular apoptosis can result in inhibiting tumor growth, inhibiting tumor metastasis, or promoting tumor apoptosis, or any combination thereof, in a patient afflicted with a tumor. For example, the tumor can be a malignant cancer. For example, the tumor can comprise breast cancer or ovarian cancer.

In various embodiments, the inhibitor can be administered to the patient in a formulation comprising a pharmaceutically acceptable excipient. Various pharmaceutically acceptable excipients are discussed below.

In various embodiments, the inhibitor can be administered to the patient orally; in other embodiments, the inhibitor can be administered to the patient parenterally. Various formulations comprising the inhibitor, adapted for oral or parenteral administration are discussed below.

Various regimens of dosing can be used in administering the apoptosis-promoting FAK inhibitor. For example, multiple administrations of the inhibitor can be provided to the patient over a period of time for a duration and at a frequency sufficient to provide a beneficial effect to the patient. In various embodiments, effective amounts of a second medicament can be administered to the patient, depending on the condition for which the patient is being treated.

In various embodiments, the invention provides a use of an inhibitor of a focal adhesion kinase inhibitor for preparation of a medicament for promoting cellular apoptosis, wherein the inhibitor comprises a compound of formula

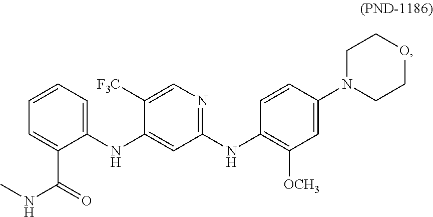

or any pharmaceutically acceptable salt thereof. In various embodiments, promoting cellular apoptosis can result in inhibiting tumor growth, or inhibiting tumor metastasis, or promoting tumor apoptosis, or any combination thereof. In various embodiments the medicament can be used for the treatment of malconditions comprising tumors, malignant or non-malignant.

It has been found by the inventors herein that PND-1186 blocks FAK Tyr-397 phosphorylation in vivo and exhibits anti-tumor efficacy in orthotopic human and murine breast carcinoma mouse tumor models. Administration of PND-1186 (100 mg/kg i.p.) resulted in sustained inhibition (>60%) of tumor FAK Tyr-397 phosphorylation for 12 hours (average 15.1 μM in plasma and 10.4 μg/g in tumors at 12 h). PND-1186 administered at 150 mg/kg p.o. bid significantly inhibited orthotopic and syngeneic breast carcinoma tumor growth and spontaneous tumor cell metastasis to lungs. Surprisingly, mice given 0.5 mg/ml PND-1186 ad libitum in their drinking water (average 1.0 μM in plasma and 0.52 μg/g in tumors) exhibited significantly decreased tumor growth and tumoral FAK-p130Cas phosphorylation. Although PND-1186 was non-cytotoxic to cells in culture, tumors from animals receiving ad libitum PND-1186 exhibited necrotic regions at the tumor core, increased TUNEL staining, and decreased leukocyte infiltrate. PND-1186 treatment reduced tumor-associated splenomegaly and tumor necrosis factor-α triggered interleukin-6 cytokine expression, indicating that FAK inhibition can impact tumors. PND-1186 may therefore be useful clinically to curb tumor growth and metastasis or progression via effects on both tumor and stromal cells, such as by promoting or inducing apoptosis of tumor cells. See: C. Walsh, et al., Oral delivery of PND-1186 FAK inhibitor decreases tumor growth and spontaneous breast to lung metastasis in pre-clinical models, Cancer Biology & Therapy (2010), 9:10, 778-790; I. Tanjoni, et al., PND-1186 FAK inhibitor selectively promotes tumor cell apoptosis in three-dimensional environments, Cancer Biology & Therapy (2010), 9:10, 764-777; which are incorporated by reference herein in their entireties.

PND-1186 has been found to have an IC₅₀ of ˜100 nM in murine and human breast carcinoma cells as determined by anti-phospho-specific immunoblotting to FAK Tyr-397. FAK inhibition does not alter c-Src, p130Cas, or paxillin tyrosine phosphorylation in cultured tumor cells. Surprisingly high concentrations (>5-fold above IC₅₀) were required for inhibition of cell growth and motility. Nonetheless, when cells were grown as colonies in soft agar or under non-adherent conditions, 100 nM PND-1186 inhibited cell proliferation, FAK-Cas phosphorylation, and induced cell death. Accordingly, low-level 0.5 mg/ml PND-1186 addition to the drinking water of mice decreased tumor burden, increased caspase 3 cleavage, and elevated tumor TUNEL staining. FAK activity therefore plays an unexpected critical role in promoting the survival of tumor cells in a three-dimensional environment, and inhibition of FAK can result in the death of tumor cells in that type of environment.

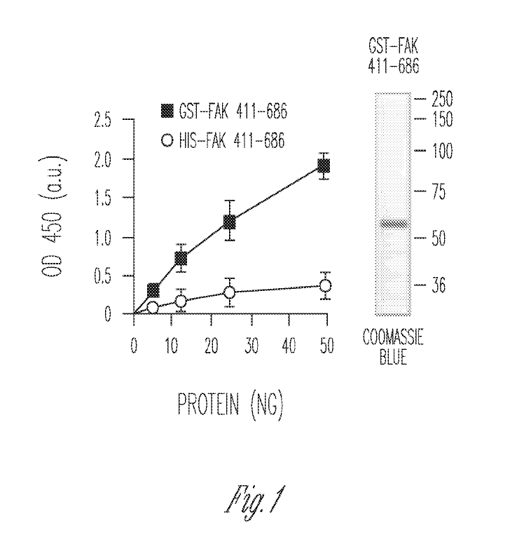

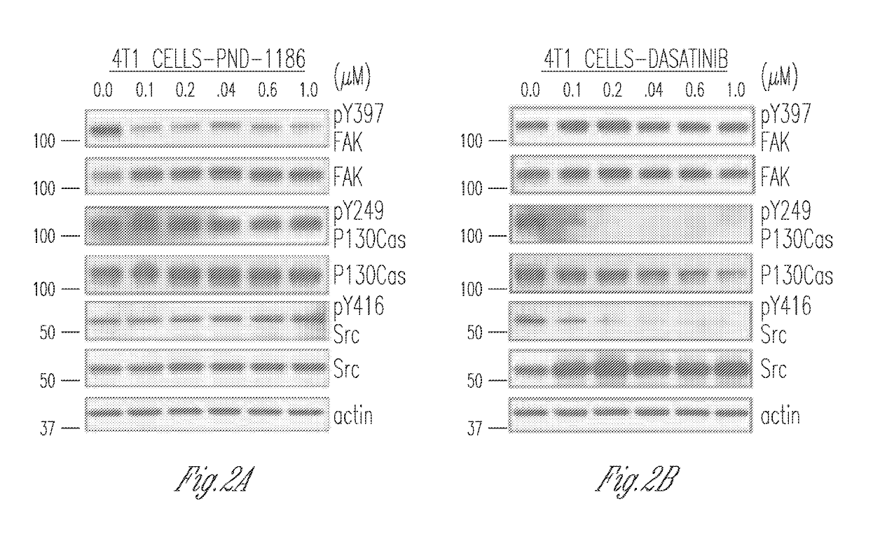

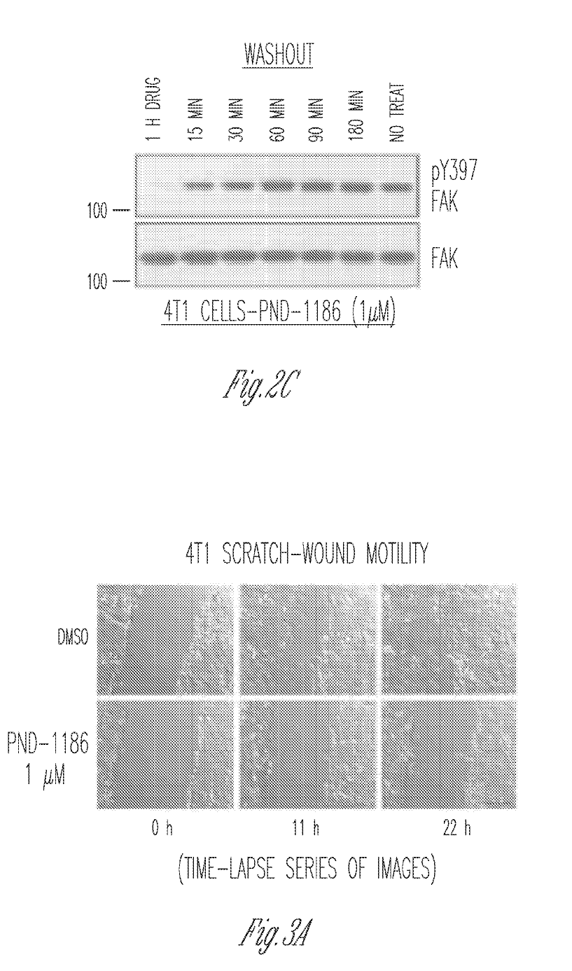

Using the recombinant FAK kinase domain as a glutathione-S-transferase (GST) fusion protein in an in vitro kinase assay (see FIG. 1), PND-1186 inhibited FAK activity with IC₅₀ of 1.5 nM. The selectivity of PND-1186 was evaluated using the Millipore KinaseProfiler Service. In this screen with recombinant protein kinases, 0.1 μM PND-1186 displayed specificity for FAK as well as Flt3 (FMS-like tyrosine kinase 3) kinase inhibition. At a higher PND-1186 levels (1 μM), FAK and Flt3 had negligible activity and other kinases including ACK1 (activated Cdc42-associated tyrosine kinase 1), Aurora-A, CDK2 (cyclin-dependent kinase 2)/cyclin A, insulin receptor (IR), Lck (lymphocyte-specific protein tyrosine kinase), and TrkA (tropomyosin-related kinase A) were inhibited greater than 50%. Flt3 expression is found in cells of hematopoietic origin and is not detectably expressed in 4T1, MDA-MB-231, or ID8 cells used herein. See Table 1, below.

TABLE 1
Kinase Profile Analyses of PND-1186

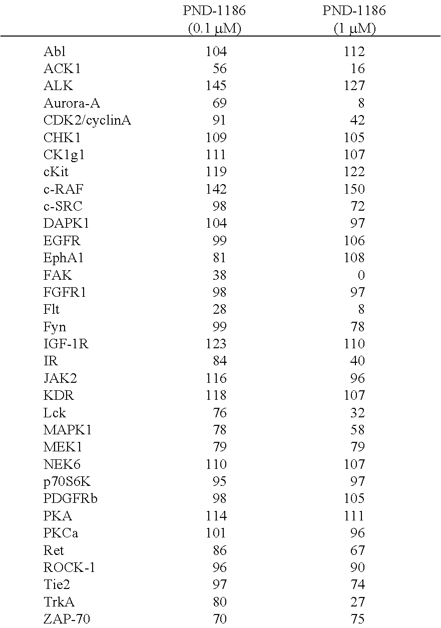

(Values are percent activity - greater than 50% inhibition highlighted)

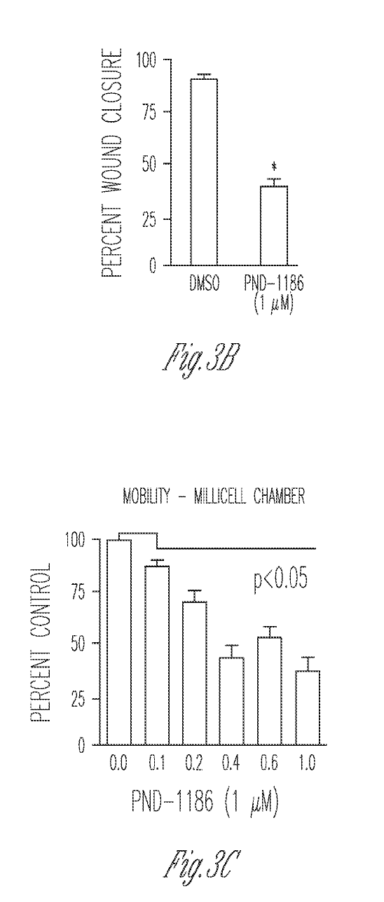

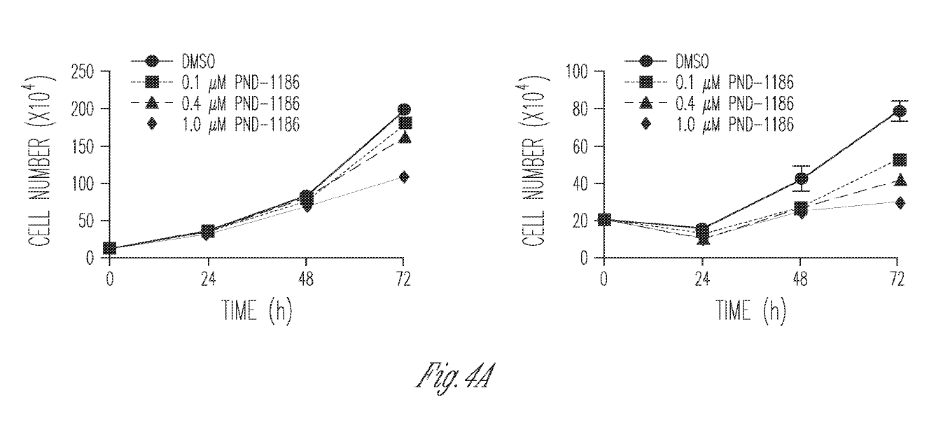

In murine 4T1 breast carcinoma cells, FAK promotes an invasive and metastatic cell phenotype. Increasing concentrations of PND-1186 (0.1 to 1.0 μM) added to 4T1 cells inhibited FAK Tyr-397 phosphorylation (pY397) and resulted in elevated levels of total FAK protein within 1 h (see FIG. 2A). Similar results were obtained by PND-1186 addition to human MD-MBA-231 breast carcinoma cells and murine ID8 ovarian carcinoma cells. The cellular IC₅₀ for FAK pY397 inhibition was determined as ˜0.1 μM PND-1186 by densitometry analyses and maximal reduction of FAK pY397 phosphorylation was ˜80% (see FIG. 2A).

PND-1186 inhibition of FAK was reversible as washout experiments showed that FAK pY397 phosphorylation fully recovered within 60 min (FIG. 2C). Surprisingly, PND-1186 addition to 1 μM did not affect c-Src activity as determined by phosphos-specific antibody reactivity to Src Tyr-416 (pY416) or p130Cas Tyr-249 (pY249) phosphorylation in adherent 4T1 cells (FIG. 2A). In contrast, when dasatinib (BMS-354825) was added to 4T1 cells (inhibiting both Abelson murine leukemia viral oncogene homolog 1, Abl and Src-family kinases), both Src pY416 and p130Cas pY249 were reduced in a dose-dependent manner (FIG. 2B). Notably, dasatinib did not affect FAK pY397 levels (FIG. 2B) and similar results were obtained using MD-MBA-231 cells or another Src inhibitor such as 4-amino-5-(4-methylphenyl)-7-(t-butyl)pyrazolo[3,4-d]-pyrimidine, commonly known as PP1. Taken together, these results show that PND-1186 potently inhibits FAK phosphorylation in a reversible manner and that Src pY416 and p130Cas pY249 phosphorylation are dependent on Src but not FAK activity in adherent 4T1 cells.

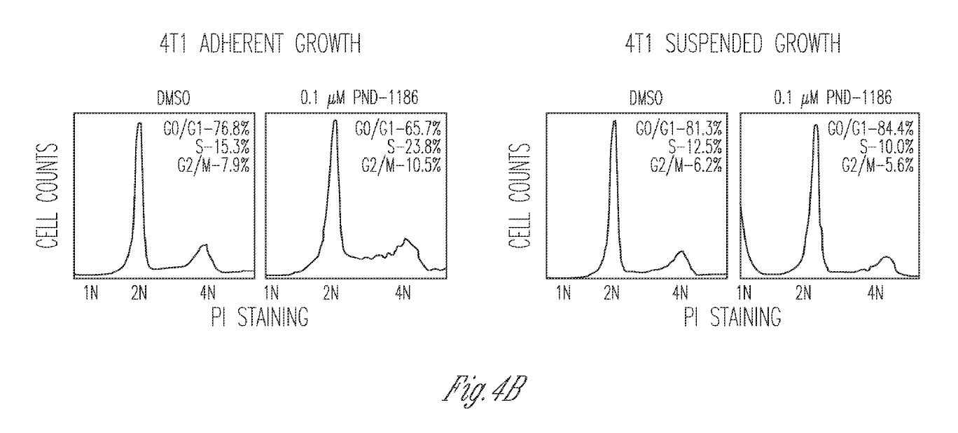

PND-1186 also inhibits 4T1 cell migration, as shown by time lapse wound healing assays were performed in the presence of DMSO (dimethyl sulfoxide, control) or 1 μM PND-1186 (FIG. 3A) for 22 h. PND-1186 prevented 4T1 cell movement and this was associated with the lack of protrusion formation and the infrequency of 4T1 edge cell separation from the collective monolayer. No evidence of 4T1 cell detachment or death was observed with 1 μM PND-1186 over 22 h and notably, cells were visualized undergoing cell division in the presence of 1.0 μM PND-1186. Quantitation of several wound regions over 22 h revealed that DMSO-treated 4T1 cells showed 89% wound closure whereas PND-1186-treated cells had only 40% closure (FIG. 3B).

To validate the wound assay results, Millicell chamber motility assays were performed with membranes coated with fibronectin and serum added as a chemotaxis stimulus (FIG. 3C). Addition of PND-1186 to the Millicell motility assay prevented 4T1 cell movement in a dose-dependent fashion with ˜50 to 60% maximal inhibition at 0.4 μM PND-1186 addition for 4 h Importantly, PND-1186 did not affect 4T1 cell adhesion to fibronectin, as did dasatinib addition at sub-micromolar levels. These results support the importance of FAK activity in promoting 4T1 cell motility.

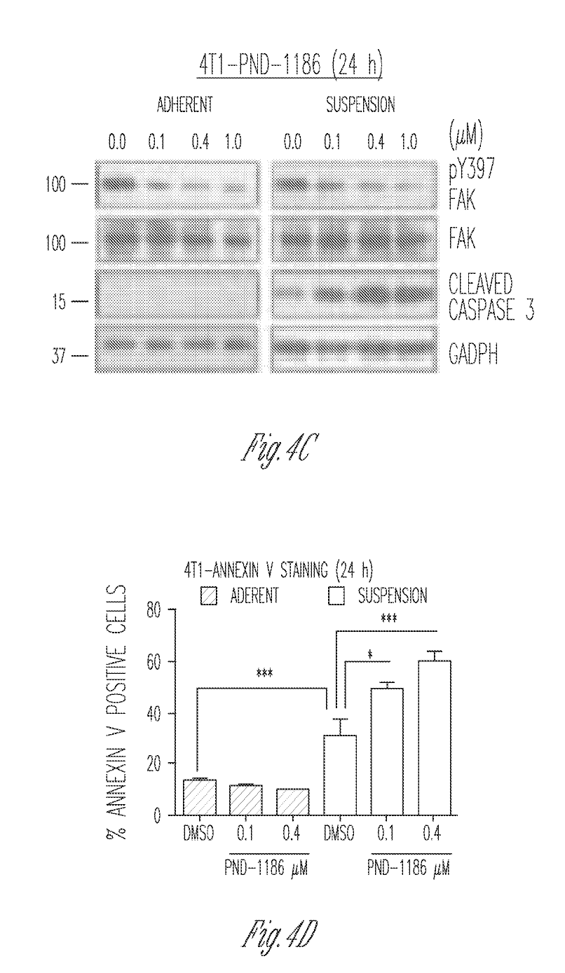

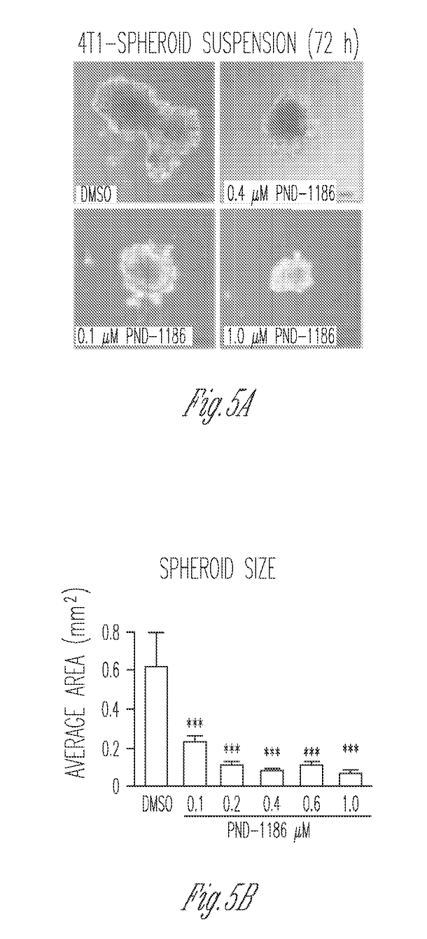

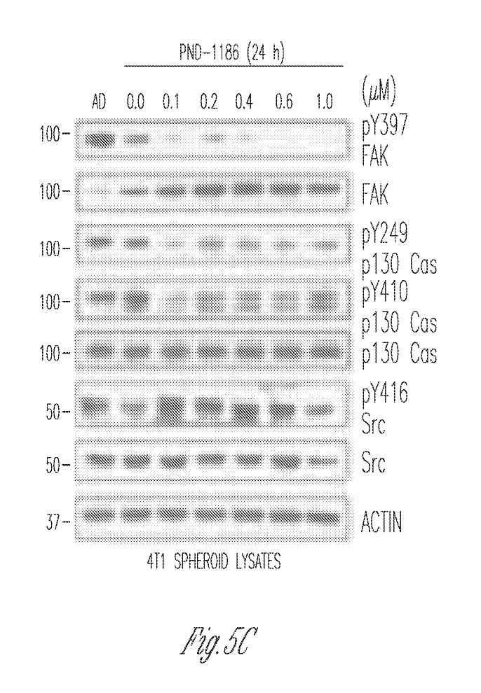

To determine effects on cell proliferation of 4T1 cells, increasing concentrations (0.1 to 1.0 μM) of PND-1186 were added to adherent or suspended (non-adherent) 4T1 cells, and total cell numbers were enumerated after 24, 48, and 72 h (FIG. 4A). In adherent cells, no differences were observed at 24 or 48 h. However at 72 h, 1.0 μM PND-1186-treated cells were decreased in number, and propidium iodide (PI) staining combined with flow cytometry analyses revealed a slight accumulation in the S and G2/M phases of the cell cycle compared to DMSO control (FIG. 4B). In suspended 4T1 cells, all concentrations of PND-1186 reduced cell numbers at 48 h and this difference was significant (p<0.001) for 0.1 μM PND-1186 at 72 h compared to DMSO (FIG. 4A). Interestingly, PI staining analyses of 1.0 μM PND-1186 treated cells in suspension did not reveal cell cycle differences (FIG. 4B). However, there was an accumulation of sub-diploid (1N) cells as detected by PI staining which is a marker of apoptosis in other cell types. These results show that PND-1186 has limited effects on cell cycle progression and effects on total cell numbers may be associated with cell death.

To determine if PND-1186 is triggering suspended 4T1 cell apoptosis, lysates of adherent and suspended 4T1 cells treated with PND-1186 for 24 h and were analyzed by immunoblotting (FIG. 4C). PND-1186 at 0.1 μM was sufficient to inhibit FAK pY397 phosphorylation in adherent and suspended cells. Notably, the detection of cleaved caspase 3 was increased in PND-1186-treated suspended cells (maximal at 0.4 μM) but was not detectable in PND-1186 treated adherent cells (FIG. 4C). Caspase 3 cleavage is associated with caspase 3 activation and is an initiator of cell apoptosis. As an independent verification of 4T1 cell apoptosis in suspension upon addition of PND-1186, cells were treated for 24 h and analyzed for annexin V binding by flow cytometry (FIG. 4D). In adherent conditions, only low levels of annexin V-positive cells were detected and this did not increase upon PND-1186 addition up to 1.0 μM. In contrast, suspended 4T1 control cells exhibited elevated annexin V staining and this was further increased to 50-60% annexin V positive staining upon 0.1 to 0.4 μM PND-1186 addition (FIG. 4D). Taken together, the triggering of 4T1 cell apoptosis under suspended conditions upon low level PND-1186 addition suggests that FAK activity may be essential for the survival of cells under anchorage-independent conditions.

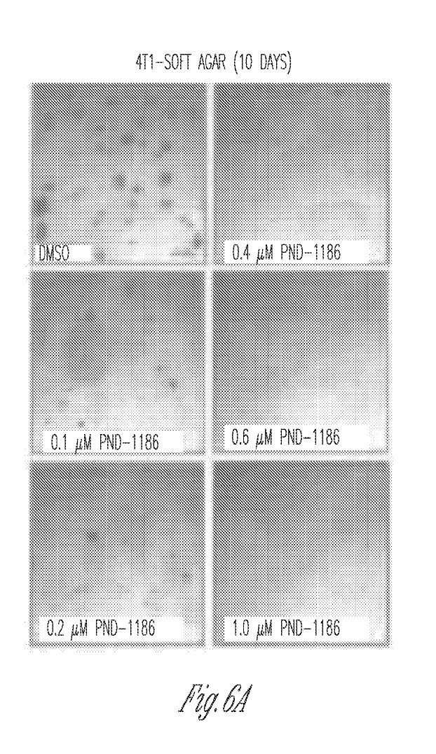

As PND-1186 selectively promotes 4T1 cell apoptosis under suspended cell conditions, 4T1 cells were cultured as 3D spheroids and increasing concentrations of PND-1186 (0.1 to 1.0 μM) were added for 72 h to determine effects on spheroid size (FIGS. 5A and B). At 0.1 μM PND-1186, there was a ˜3-fold reduction in average spheroid size and maximal effects were observed at 0.2 μM PND-1186. To date, no other FAK inhibitor known to Applicants has shown maximal inhibition of a biological response at sub-micromolar levels.

To determine specificity of PND-1186 FAK inhibition in 4T1 cellular spheroids, immunoblotting was performed (FIG. 5C). The total level of FAK pY397 phosphorylation was reduced under 3D spheroid compared to adherent 4T1 conditions. No differences were found for p130Cas pY249 or p130Cas pY410 phosphorylation in adherent versus 3D spheroid conditions (FIG. 5C, lanes 1 and 2). However, 0.1 μM PND-1186 potently inhibited FAK pY397, p130Cas pY249, and p130Cas pY410 phosphorylation in 4T1 spheroids (FIG. 5C, lanes 3). Increasing PND-1186 addition resulted in elevated total FAK levels, no change in p130Cas expression, and sustained inhibition of FAK and p130Cas tyrosine phosphorylation (FIG. 5C). There was no change in either Src pY416 or Src expression levels in adherent, spheroid, or PND-1186-treated spheroid 4T1 cells (FIG. 5C). Importantly, the inhibition of p130Cas phosphorylation by PND-1186 in 4T1 spheroids differs from the lack of PND-1186 effects on 4T1 cells as a two-dimensional (2D) monolayer. FAK phosphorylation of targets such as p130Cas can facilitate the survival of 4T1 cells in 3D conditions.

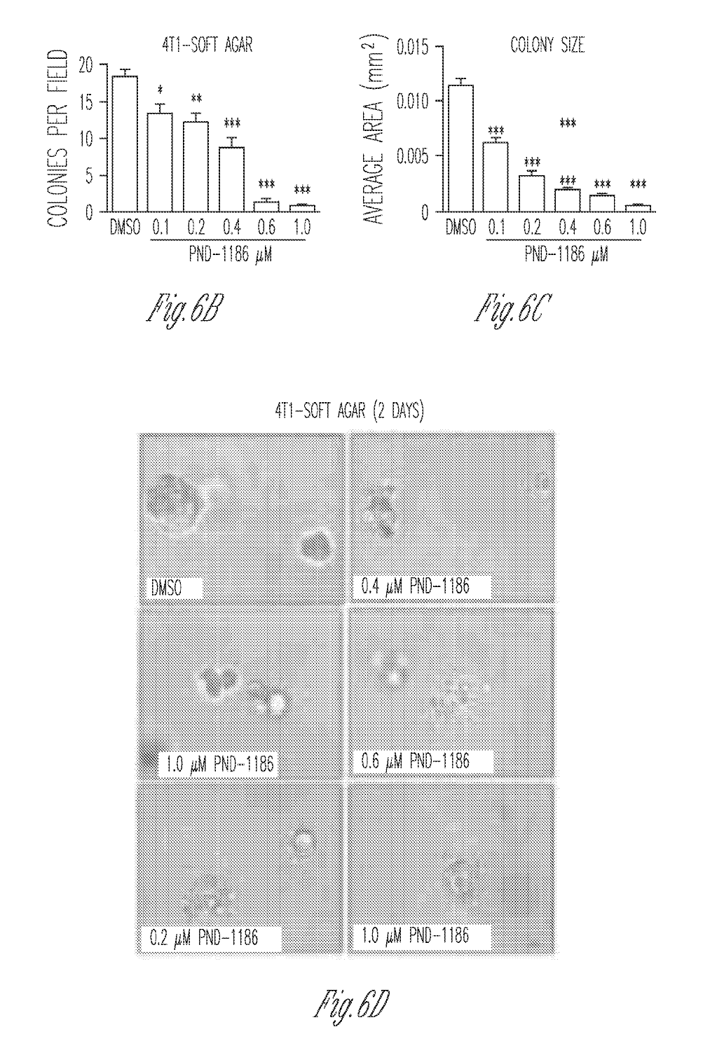

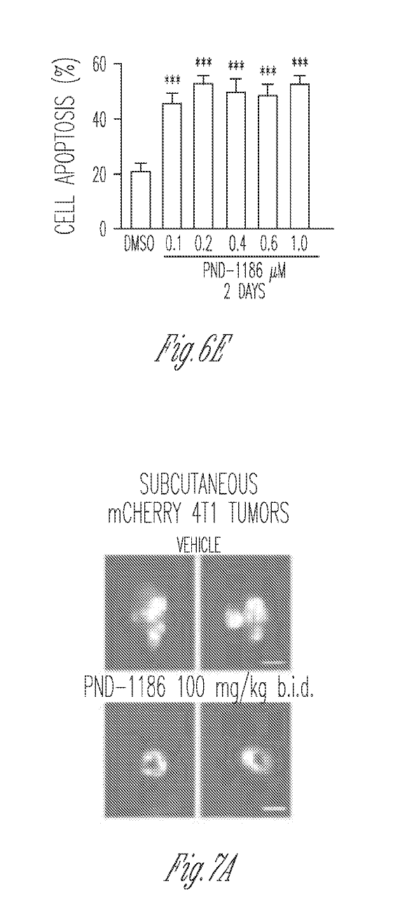

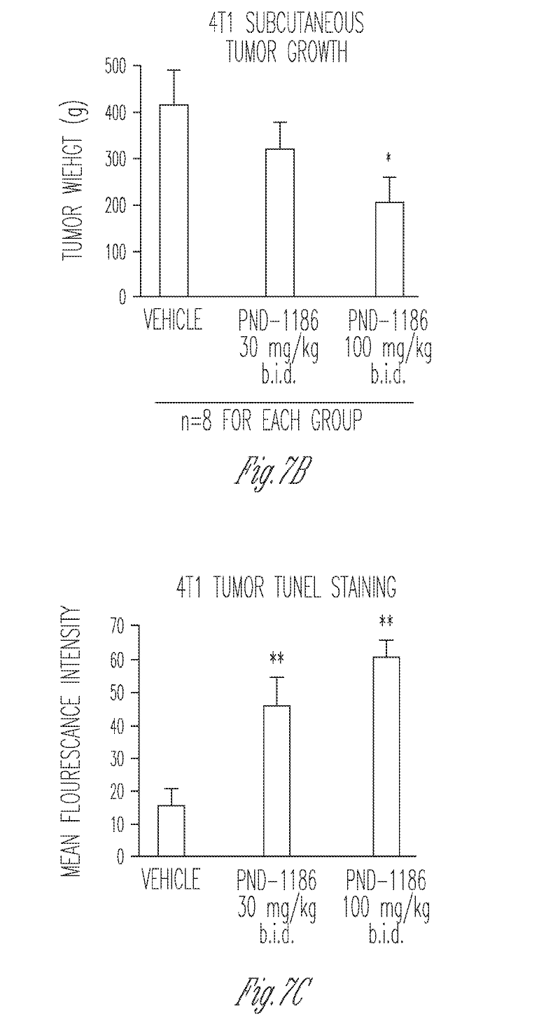

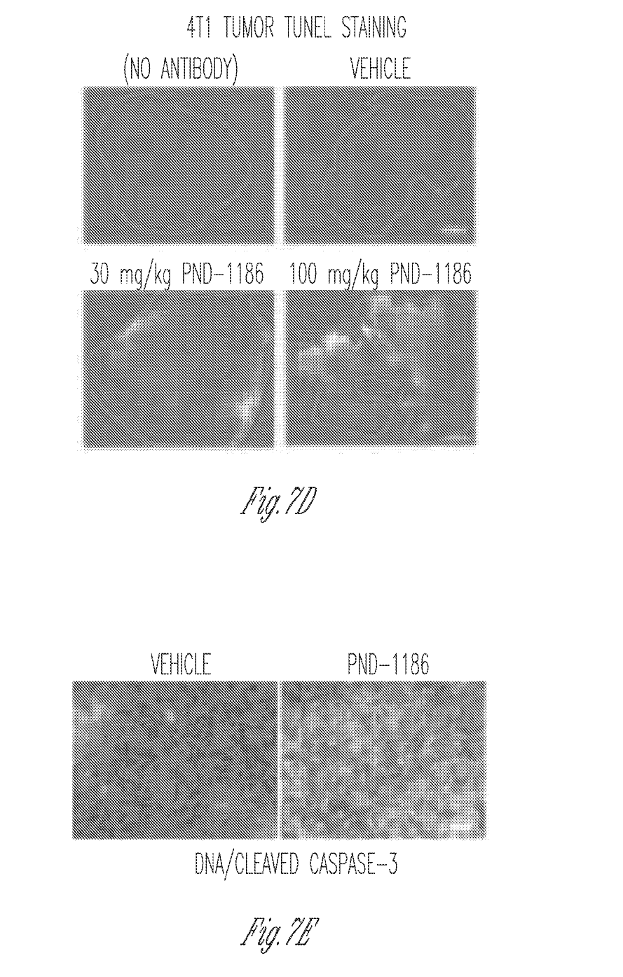

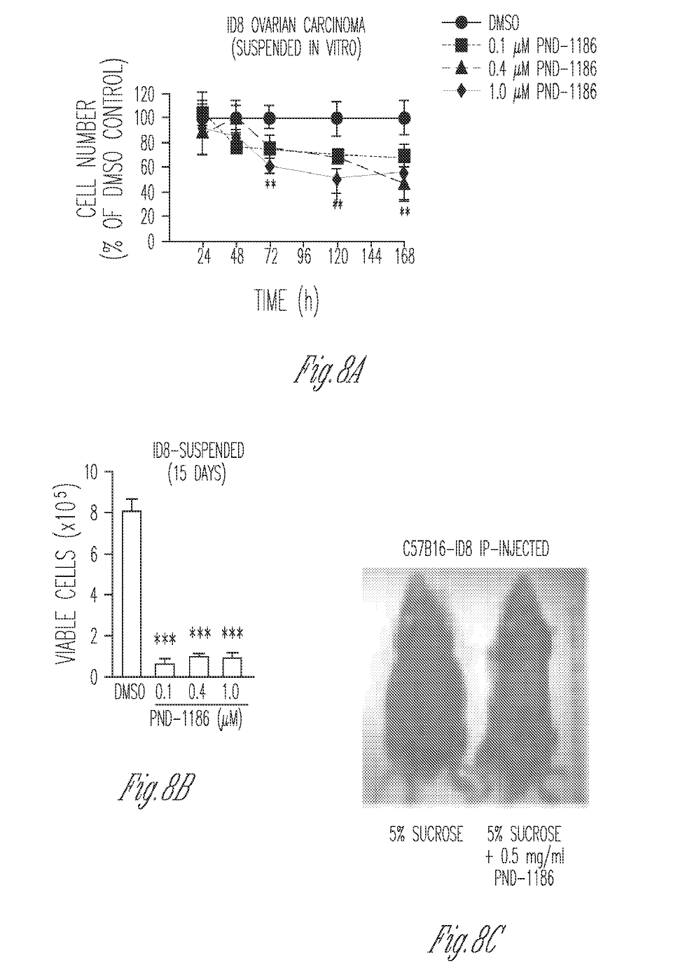

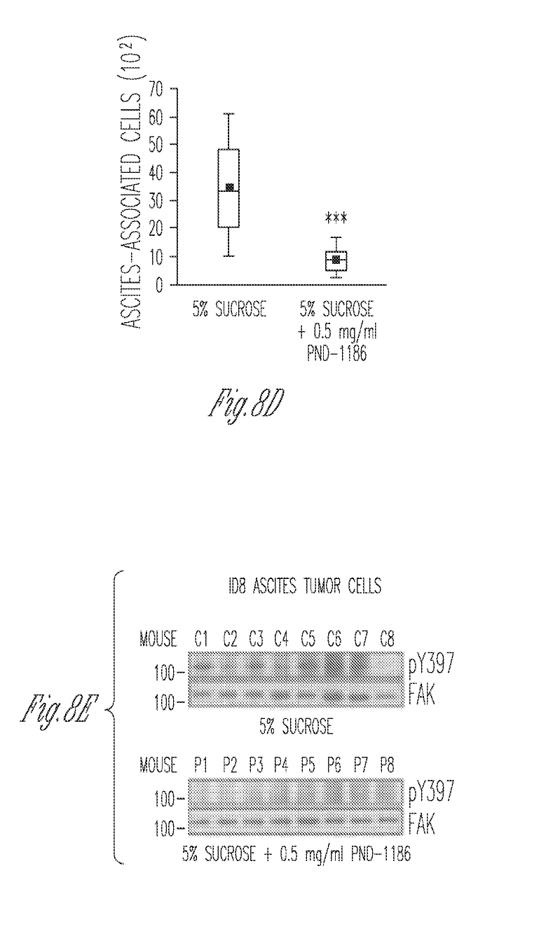

4T1 cells were grown as colonies in soft agar and the effects of PND-1186 addition evaluated (FIG. 6). By 10 days, PND-1186 addition inhibited both the total number and size of 4T1 soft agar colonies in a dose-dependent manner (FIG. 6A-C). Similar results were obtained when PND-1186 was added 4 days after the establishment of 4T1 cells in soft agar (FIG. 7). At 0.2 μM PND-1186, 4T1 soft agar colony size was inhibited 77% (FIG. 6C) and this corresponded to increased 4T1 cell apoptosis (>50%) as determined by membrane blebbing and cell shrinkage (FIGS. 6D and E). Taken together, our results support the hypothesis that PND-1186 does not work as a general cytotoxic drug, but selectively and potently interferes with the survival of cells in a 3D environment.

To determine the sensitivity of 4T1 tumor growth to PND-1186 administration, mCherry fluorescently-labeled 4T1 cells were grown subcutaneously in BALB/c mice (FIG. 7). After allowing eight days for primary tumor establishment, vehicle or PND-1186 at 30 mg/kg or at 100 mg/kg was administered every 12 h (twice-daily, b.i.d.) for 5 days after which time, mCherry 4T1 tumors were visualized in situ followed by extraction and weighing (FIGS. 7A and B). Whereas vehicle-treated 4T1 tumors were brightly fluorescent, generally multi-lobed and had become invasive to the surrounding tissues, tumors in mice treated with 100 mg/kg PND-1186 contained dark non-fluorescent centers, were generally rounded, and were loosely adherent to sub-dermal tissues (FIG. 7A). 100 mg/kg PND-1186 treatment significantly reduced final 4T1 tumor weight 2-fold (n=8, p<0.05) whereas 30 mg/kg PND-1186 slightly reduced final tumor weight but was not significantly different compared to control (n=8, p>0.05). To determine if the loss of mCherry fluorescence in the center of 100 mg/kg PND-1186 tumors was associated with increased cell apoptosis, medial sections were analyzed by TUNEL (FIGS. 7C and D) and anti-cleaved caspase 3 (FIG. 7E) staining. Both 30 and 100 mg/kg administration of PND-1186 significantly increased tumor TUNEL staining compared to vehicle-treated controls (FIG. 7D). As elevated cleaved caspase-3 staining was also found in the tumors of PND-1186-treated mice (FIG. 7E), these results parallel our in vitro analyses and show that PND-1186 promotes apoptosis of 4T1 cells in 3D conditions resulting in the inhibition of tumor growth in vivo.

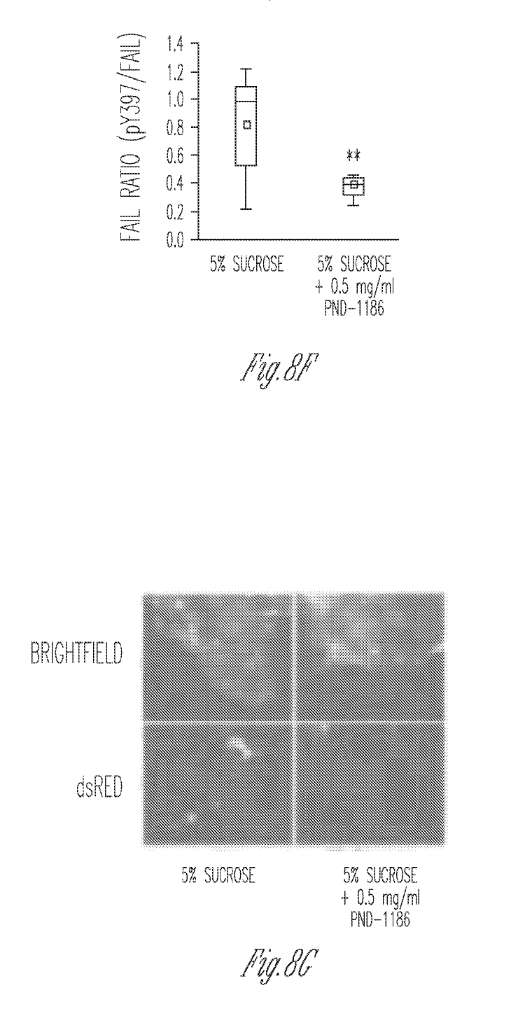

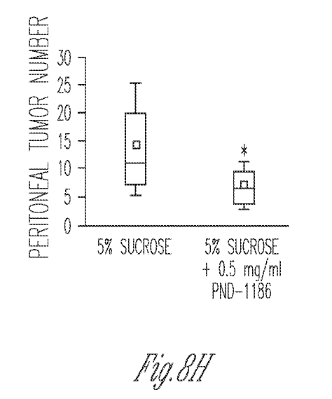

During ovarian carcinoma tumor cell progression, cells can dissociate from the primary tumor and grow as multi-cellular spheroids within the peritoneal space. As PND-1186 selectively promotes 4T1 breast carcinoma apoptosis in 3D environments, PND-1186 effects on murine ID8 ovarian carcinoma cell growth were evaluated in vitro and in vivo (FIG. 8). In suspended cell culture as spheroids, 0.1, 0.4, and 1.0 μM PND-1186 significantly inhibited ID8 cell number at 72 h (FIG. 8A) and resulted in a dramatic reduction in viable cells after 15 days (FIG. 8B). To determine if low levels of PND-1186 could affect ID8 growth in vivo, dsRed fluorescently-labeled ID8 cells were intraperitoneally-injected into C57B16 mice and after 11 days, mice were provided 5% sucrose (control) or 0.5 mg/ml PND-1186 in 5% sucrose in lieu of drinking water on an ad libitum basis. No adverse effects and no body weight loss were noted with PND-1186 administration. After 30 treatment days, mice with PND-1186 in the drinking water did not exhibit swollen abdomens as did control mice (FIG. 8C). This corresponded with a lower number of ascites-associated cells (FIG. 8D) and the >2-fold inhibition of FAK pY397 by 0.5 mg/ml PND-1186 administration compared to 5% sucrose controls (FIGS. 8E and F). In addition to inhibiting ascites-associated ID8 spheroid growth, PND-1186-treated mice showed significantly fewer tumor nodules within the peritoneal space as detected by in vivo dsRed fluorescence imaging (FIGS. 8G and H). These results show that low level PND-1186 administration inhibits the growth of ovarian carcinoma cells in vitro and in vivo. The selective effects on PND-1186 in promoting apoptosis of cells in three dimensional environments points to a novel role for FAK activity in generating anchorage-independent survival signals.

Oral PND-1186 dosing provided significant anti-tumor and anti-metastatic effects in two different (4T1 and MDA-MB-231) orthotopic breast carcinoma mouse tumor models without animal morbidity, death or weight loss. PND-1186 significantly decreased tumor-associated inflammatory cell infiltration and splenomegaly in mice with syngeneic 4T1 tumors, suggesting PND-1186 can reduce tumor-associated inflammation.

PND-1186 pharmacokinetics (PK) were determined in Balb/c mice following intravenous (i.v.), intraperitoneal (i.p.), and oral (p.o.) administration (Table 2, below). PND-1186 displayed a multi-exponential decay with a terminal half life (t_{1/2}) of 1.72 hours after i.v. injection. Following i.p. and p.o. dosing, PND-1186 was rapidly absorbed) with terminal half lives (t_{1/2}) of 2.15 to 2.65 h, and bioavailability (% F) from 14.8 to 42.2%. PND-1186 bioavailability was greater upon intraperitoneal versus oral dosing PND-1186 plasma concentrations, maximum concentration (Cₘₐₓ), and the area under the plasma concentration-time curve (AUC) from time zero to infinity (0-inf) increased in a linear fashion as a function of dose.

TABLE 2
PND-1186 pharmacokinetic (PK) parameters after intravenous (i.v.),
intraperitoneal (i.p.), oral (p.o.), and ad libitum dosing in mice.

| | C_{MAX} | T_{MAX} | C_{SS} | t_{1/2} | AUC(0-inf) | Vd | Cl |
|---|---|---|---|---|---|---|---|
| Dose | (μM) | (h) | (μM) | (h) | (ng · h/mL) | (ml/kg) | (ml/h/kg) |
| PND-1186 2 mg/kg i.v. | — | — | — | 1.72 | 6,960 | 714 | 287 |
| PND-1186 30 mg/kg i.p. | 34.76 | 0.25 | — | 2.27 | 32,500 | — | — |
| PND-1186 100 mg/kg i.p. | 117.10 | 0.50 | — | 2.65 | 147,000 | — | — |
| PND-1186 150 mg/kg p.o. | 13.98 | 4.00 | — | 2.15 | 77,400 | — | — |
| PND-1186 0.5 mg/kg ad libitum | — | — | 1.16 | — | 13.1 | — | — |

PK parameters listed include the observed maximum plasma concentration (Cₘₐₓ) and time to maximum concentration (Tₘₐₓ) after i.p. or p.o. dosing, area under the plasma concentration-time curve from time zero to infinity AUC(0-inf), volume of distribution (V_{d}), systemic clearance (Cl), log linear terminal half life (t_{1/2},) and the bioavailability (% F). PK analyses were performed by non-compartmental analysis using model 200 for i.p. and p.o. and model 201 for the i.v. in WinNonlin Professional 4.1 (Pharsight Corp., Mountain View, CA).

To determine if PND-1186 affected FAK and p130Cas in solid tumors, subcutaneous 4T1 breast carcinoma tumors were established and a single i.p. injection of vehicle (50% PEG400 in PBS) or PND-1186 was administered. For 100 mg/kg PND-1186, maximal plasma levels (117 μM) were reached within 30 min and maximal PND-1186 in tumors (16.1 μg/g) was achieved within 1 h and maintained up to 12 h (with plasma levels at 1.1 μM at 12 h). 100 mg/kg PND-1186 resulted in sustained inhibition (>60%) of tumor FAK Tyr-397 phosphorylation (pY397 FAK) for 12 h and significantly reduced p130Cas Tyr-410 phosphorylation (pY410Cas) by 3 h. Similar results were obtained when using phospho-specific antibodies to pTyr-249 of p130Cas. For 30 mg/kg PND-1186, maximal plasma levels (35 μM) were reached within 15 min and maximal PND-1186 in tumors (0.75 μg/g) was achieved within 1 h. This was sufficient to inhibit FAK pY397 phosphorylation for 3 to 6 h after which time tumor PND-1186 levels fell to 0.04 μg/g by 12 h (with plasma levels at 0.1 μM) at which point tumor FAK pY397 phosphorylation was not significantly inhibited. These results show that PND-1186 inhibits FAK and p130Cas tyrosine phosphorylation in tumors in a dose-dependent manner in vivo and that plasma levels at or above 1 μM are sufficient to promote tumor-associated FAK inhibition.

Oral bioavailability of PND-1186 in water is less than when administered intraperitoneally (Table 2, above). As 150 mg/kg oral dose of PND-1186 resulted in maximal plasma level of ˜14 μM by 4 h and a sustained plasma level of PND-1186 above 3 μM for 12 h, this oral (p.o) twice-daily (b.i.d.) dose was tested for anti-tumor efficacy using orthotopic implanted mCherry-fluorescent 4T1 tumor cells. By Day 7, 150 mg/kg PND-1186 significantly reduced tumor volume compared to vehicle control. By 16 days, 150 mg/kg PND-1186 reduced final tumor volume 3-fold and final tumor weight was reduced 3.1-fold compared to vehicle control without affects on total body weight Analyses of primary breast fat pad 4T1 tumors revealed a high number of blood vessels as detected by anti-CD31 staining. Although previous studies with lung carcinoma xenografts showed reduced tumor microvessel density after PF-562,271 administration, no major vascular differences were observed in PND-1186-treated 4T1 orthotopic tumors as determined by anti-CD31 staining. To determine a potential molecular mechanism to account for the smaller size of PND-1186-treated 4T1 tumors, medial sections were analyzed by deoxynucleotidyl transferase dUTP nick end labeling (TUNEL). Mice administered PND-1186 exhibited 2.8-fold increased TUNEL staining in breast fat pad tumors compared to vehicle-treated controls. Thus, increased tumor cell apoptosis can be a mechanism responsible for the inhibition of tumor growth by PND-1186.

4T1 tumor sections were analyzed for CD45 staining, a common marker present on macrophages and other hematopoietic cells. In untreated and vehicle-treated mice, there was abundant number of CD45-positive cells present within 4T1 primary tumors. Mice treated with 150 mg/kg PND-1186 exhibited a 2.8-fold decrease in CD45 tumor-associated staining, supportive of reduced immune cell infiltration into 4T1 tumors upon PND-1186 treatment.

To determine if this was localized or a systemic response, spleen size was analyzed in normal Balb/c mice or tumor-bearing mice treated with vehicle or PND-1186. Spleens from vehicle-treated mice weighed >2-fold more than PND-1186-treated mice. Notably, spleens from PND-1186-treated mice were healthy and indistinguishable from non-treated, non-tumor bearing mice. As splenomegaly is due in part to increased inflammatory cytokine production, 4T1 cells in culture were stimulated by tumor necrosis factor-α (TNFα) addition and interleukin-6 (IL-6) cytokine production was measured by an enzyme-linked immunosorbent assay (ELISA). TNFα triggered >4-fold increase in 4T1 IL-6 production and PND-1186 addition (0.25 to 1.0 μM) inhibited IL-6 release in a dose-dependent manner. The anti-inflammatory effects of PND-1186 treatment can act to limit 4T1 tumor progression.

4T1 tumors are used as a model of late-stage breast cancer progression. See, for example, Heppner G H, Miller F R, Shekhar P M., Nontransgenic models of breast cancer, Breast Cancer Res 2000, 2:331-4. 4T1 cells implanted into the breast fat pad will intravasate into the blood circulation and form pulmonary metastases within 7 to 10 days. As PND-1186 inhibits both 4T1 tumor growth and associated inflammation, the metastatic distribution of mCherry-fluorescent 4T1 cells was determined after orthotopic breast fat pad injection and PND-1186 (150 mg/kg p.o., b.i.d.) treatment for 15 days. Direct visualization of mCherry fluorescence from dorsal and ventral lung images was quantified, the number of lung metastases counted, and distributions grouped as negligible, moderate, or high. For vehicle control mice, the majority had moderate and high lung metastatic burden (7/12) whereas in PND-1186 mice, the majority had negligible lung metastases (7/12) and no mice with a high metastatic burden. These findings were confirmed by hematoxylin and eosin (H&E) staining of lung sections that showed detectable 4T1 lung metastases in control but not PND-1186-treated mice. Thus, the small molecule FAK inhibitor PND-1186 can interrupt the processes of spontaneous breast cancer metastasis.

To determine the effects on tumor growth, ad libitum PND-1186 oral administration to mice was initiated 48 h after mCherry-4T1 orthotopic tumor implantation. By Day 13, tumor size was significantly different as determined by caliper measurements and by Day 22, PND-1186 administration inhibited final tumor mass >1.8 fold without toxicity or weight loss. In immunoblotting analyses of primary 4T1 tumors, ad libitum PND-1186 administration was sufficient to inhibit both FAK pY397 and p130Cas pY410 phosphorylation. Ad libitum PND-1186 also inhibited pY118 paxillin phosphorylation but not pY416 Src nor pY402 Pyk2, pS473 Akt, or pT308 Akt phosphorylation in tumors. Spleen comparisons revealed that ad libitum PND-1186 treated mice were of normal size whereas control mice had enlarged spleens. Control mice receiving 5% sucrose exhibited a moderate to high lung metastatic burden (9/11) whereas the majority of ad libitum PND-1186 mice had a negligible to moderate lung metastatic burden (13/15). Thus, low level PND-1186 administration is efficacious in slowing 4T1 tumor progression in vivo.

To extend the 4T1 findings to human breast carcinoma, MDA-MB-231 cells containing activating mutations in K-Ras and B-Raf were implanted in the breast fat pad of NOD/severe combined immunodeficiency (SCID) mice. After 12 days, when tumors became palpable, 0.5 mg/ml PND-1186 or control 5% sucrose was provided ad libitum as drinking water. By experimental Day 27 (15 days of PND-1186 administration), control tumors were significantly larger as determined by caliper measurements and at Day 70, ad libitum PND-1186 administration resulted in a >5-fold decrease in final tumor weight. Low level PND-1186 treatment was sufficient to significantly reduce FAK pY397 phosphorylation in MDA-MB-231 tumors. To determine the effect on spontaneous MDA-MB-231 metastasis, lungs from NOD/SCID mice were sectioned, H&E-stained, and micro-metastases enumerated. Control mice exhibited detectable metastasis and PND-1186 ad libitum treatment reduced the number of metastatic lung lesions >3.5-fold.

As PND-1186 treatment reduces primary tumor size which can affect a number of factors influencing tumor cell metastasis, experimental metastasis assays were performed by tail vein injection of mCherry fluorescent protein-expressing MDA-MB-231 cells. Mice were pre-administered 150 mg/kg PND-1186 or water (vehicle) p.o. and the accumulation or lodging of tumor cells within lung capillaries after 1 or 6 h was quantified by fluorescent imaging. At both 1 and 6 h, PND-1186 significantly inhibited the accumulation of tumor cells in the lungs. As blood plasma levels of PND-1186 are likely above 1 μM in the experimental metastasis assay, MDA-MB-231 apoptosis was analyzed in vitro by incubating cells in suspension with PND-1186. As determined by annexin V binding and quantified by flow cytometry, concentrations up to 10 μM PND-1186 did not promote increased MDA-MB-231 apoptosis within 6 h. Thus, oral PND-1186 administration decreases FAK tyrosine phosphorylation in vivo resulting in robust anti-tumor and anti-metastatic activity using two different orthotopic breast carcinoma models.

### Pharmaceutical Compositions and Administration Thereof for Methods of the Invention

Another aspect of an embodiment of the invention provides compositions of the compounds of the invention, alone or in combination with another medicament, for administration of the small molecule FAK inhibitors to patients, human or otherwise. Compositions containing a compound of the invention can be prepared by conventional techniques, e.g. as described in Remington: The Science and Practice of Pharmacy, 19th Ed., 1995, or later versions thereof, incorporated by reference herein. The compositions can appear in conventional forms, for example capsules, tablets, aerosols, solutions, suspensions or topical applications.

Typical compositions include a compound of the invention and a pharmaceutically acceptable excipient which can be a carrier or a diluent. For example, the active compound will usually be mixed with a carrier, or diluted by a carrier, or enclosed within a carrier which can be in the form of an ampoule, capsule, sachet, paper, or other container. When the active compound is mixed with a carrier, or when the carrier serves as a diluent, it can be solid, semi-solid, or liquid material that acts as a vehicle, excipient, or medium for the active compound. The active compound can be adsorbed on a granular solid carrier, for example contained in a sachet. Some examples of suitable carriers are water, salt solutions, alcohols, polyethylene glycols, polyhydroxyethoxylated castor oil, peanut oil, olive oil, gelatin, lactose, terra alba, sucrose, dextrin, magnesium carbonate, sugar, cyclodextrin, amylose, magnesium stearate, talc, gelatin, agar, pectin, acacia, stearic acid or lower alkyl ethers of cellulose, silicic acid, fatty acids, fatty acid amines, fatty acid monoglycerides and diglycerides, pentaerythritol fatty acid esters, polyoxyethylene, hydroxymethylcellulose and polyvinylpyrrolidone. Similarly, the carrier or diluent can include any sustained release material known in the art, such as glyceryl monostearate or glyceryl distearate, alone or mixed with a wax.

The formulations can be mixed with auxiliary agents which do not deleteriously react with the active compounds. Such additives can include wetting agents, emulsifying and suspending agents, salt for influencing osmotic pressure, buffers and/or coloring substances preserving agents, sweetening agents or flavoring agents. The compositions can also be sterilized if desired.

The route of administration can be any route which effectively transports the active compound of the invention to the appropriate or desired site of action, such as oral, nasal, pulmonary, buccal, subdermal, intradermal, transdermal or parenteral, e.g., rectal, depot, subcutaneous, intravenous, intraurethral, intramuscular, intranasal, ophthalmic solution or an ointment, the oral route being preferred.

If a solid carrier is used for oral administration, the preparation can be tabletted, placed in a hard gelatin capsule in powder or pellet form or it can be in the form of a troche or lozenge. If a liquid carrier is used, the preparation can be in the form of a syrup, emulsion, soft gelatin capsule or sterile injectable liquid such as an aqueous or non-aqueous liquid suspension or solution.

Injectable dosage forms generally include aqueous suspensions or oil suspensions which can be prepared using a suitable dispersant or wetting agent and a suspending agent Injectable forms can be in solution phase or in the form of a suspension, which is prepared with a solvent or diluent. Acceptable solvents or vehicles include sterilized water, Ringer's solution, or an isotonic aqueous saline solution. Alternatively, sterile oils can be employed as solvents or suspending agents. Preferably, the oil or fatty acid is non-volatile, including natural or synthetic oils, fatty acids, mono-, di- or tri-glycerides.

For injection, the formulation can also be a powder suitable for reconstitution with an appropriate solution as described above. Examples of these include, but are not limited to, freeze dried, rotary dried or spray dried powders, amorphous powders, granules, precipitates, or particulates. For injection, the formulations can optionally contain stabilizers, pH modifiers, surfactants, bioavailability modifiers and combinations of these. The compounds can be formulated for parenteral administration by injection such as by bolus injection or continuous infusion. A unit dosage form for injection can be in ampoules or in multi-dose containers.

The formulations of the invention can be designed to provide quick, sustained, or delayed release of the active ingredient after administration to the patient by employing procedures well known in the art. Thus, the formulations can also be formulated for controlled release or for slow release.

Compositions contemplated by the present invention can include, for example, micelles or liposomes, or some other encapsulated form, or can be administered in an extended release form to provide a prolonged storage and/or delivery effect. Therefore, the formulations can be compressed into pellets or cylinders and implanted intramuscularly or subcutaneously as depot injections. Such implants can employ known inert materials such as silicones and biodegradable polymers, e.g., polylactide-polyglycolide. Examples of other biodegradable polymers include poly(orthoesters) and poly(anhydrides).

For nasal administration, the preparation can contain a compound of the invention, dissolved or suspended in a liquid carrier, preferably an aqueous carrier, for aerosol application. The carrier can contain additives such as solubilizing agents, e.g., propylene glycol, surfactants, absorption enhancers such as lecithin (phosphatidylcholine) or cyclodextrin, or preservatives such as parabens.

For parenteral application, particularly suitable are injectable solutions or suspensions, preferably aqueous solutions with the active compound dissolved in polyhydroxylated castor oil.

Tablets, dragees, or capsules having talc and/or a carbohydrate carrier or binder or the like are particularly suitable for oral application. Preferable carriers for tablets, dragees, or capsules include lactose, corn starch, and/or potato starch. A syrup or elixir can be used in cases where a sweetened vehicle can be employed.

A typical tablet that can be prepared by conventional tabletting techniques can contain:

| Core: | Active compound (as free | 250 | mg |
|---|---|---|---|
| | compound or salt thereof) | | |
| | Colloidal silicon dioxide (Aerosil) ® | 1.5 | mg |
| | Cellulose, microcryst. (Avicel) ® | 70 | mg |
| | Modified cellulose gum (Ac-Di-Sol) ® | 7.5 | mg |
| | Magnesium stearate | Ad. | |
| Coating: | HPMC approx. | 9 | mg |
| | *Mywacett 9-40 T approx. | 0.9 | mg |

*Acylated monoglyceride used as plasticizer for film coating.

The compounds of the invention are effective over a wide dosage range. For example, in the treatment of adult humans, dosages from about 0.05 to about 5000 mg, preferably from about 1 to about 2000 mg, and more preferably between about 2 and about 2000 mg per day can be used. A typical dosage is about 10 mg to about 1000 mg per day. In choosing a regimen for patients it can frequently be necessary to begin with a higher dosage and when the condition is under control to reduce the dosage. The exact dosage will depend upon the activity of the compound, mode of administration, on the therapy desired, form in which administered, the subject to be treated and the body weight of the subject to be treated, and the preference and experience of the physician or veterinarian in charge.

Generally, the compounds of the invention are dispensed in unit dosage form including from about 0.05 mg to about 1000 mg of active ingredient together with a pharmaceutically acceptable carrier per unit dosage.

Usually, dosage forms suitable for oral, nasal, pulmonal or transdermal administration include from about 125 μg to about 1250 mg, preferably from about 250 μg to about 500 mg, and more preferably from about 2.5 mg to about 250 mg, of the compounds admixed with a pharmaceutically acceptable carrier or diluent.

Dosage forms can be administered daily, or more than once a day, such as twice or thrice daily. Alternatively dosage forms can be administered less frequently than daily, such as every other day, or weekly, if found to be advisable by a prescribing physician.

### Examples

Chemical Compound: PND-1186 was synthesized and used as an HCl salt as described in PCT patent application number PCT/US2008/003205, filed Mar. 10, 2008, and published as WO 2008/115369. For in vivo assays, PND-1186 was dissolved in water (solubility=22 mg/ml).

Baculovirus FAK catalytic domain and in vitro kinase assays: The FAK catalytic domain region (411-686) was generated by polymerase chain reaction using the primers 5′-cgatcgaattctcgaccagggattatgagattca-3′ 5′-tagctgtcgacttactgcaccttctcctcctccagg-3′, cloned into pGEX4T as a fusion with GST, and moved into the pAcG2T baculovirus expression vector (Pharmingen, Baculogold). Virus clones were identified by plaque assays and amplified. For protein expression, SF9 cells were transduced at a multiplicity of infection of 2-5 pfu/cell and cultured at 27° C. for 48 h. Glutathione agarose affinity chromatography were used to purify GST-FAK (411-686) followed by size fractionation using hiload 16/60 Superdex chromatography (GE Healthcare). Protein was concentrated and stored frozen in 50 mM Tris pH 8.0, 150 mM NaCl, 1 mM Na orthovanadate, 0.5 mM EDTA, 0.5 mM EGTA, 0.1% β-mercaptoethanol, and 20% glycerol. Purity was estimated at >90% by SDS-PAGE. GST-FAK in vitro kinase activity was measured and compared to His-tagged FAK 411-686 (Millipore) using the K-LISA screening kit (Calbiochem) and poly(Glu:Tyr) (4:1) copolymer (P0275, Sigma) as a substrate immobilized on microtiter plates. IC₅₀ values were determined with various concentrations of test compounds in a buffer containing 50 μM ATP and 10 mM MnCl₂, 50 mM HEPES (pH 7.5), 25 mM NaCl, 0.01% BSA, and 0.1 mM Na orthovanadate for 5 min at room temperature. Serial diluted compounds at ½-Log concentrations (starting at 1 μM) were tested in triplicate. Substrate phosphorylation was measured using horseradish peroxidase-conjugated anti-pTyr antibodies (PY20, Santa Cruz Biotechnology) with spetrophotometic color quantitation. IC₅₀ values were determined using the Hill-Slope Model. Kinase selectivity profiling was performed by using the KinaseProfiler service (Millipore).

Reagents and cells: Antibodies to β-actin (AC-17) were from Sigma-Aldrich. Antibodies to Src (Src-2) and Akt were from Santa Cruz Biotechnology. Antibodies to FAK (4.47) were from Millipore. Site and phospho-specific antibodies to pY249 p130Cas, pY410 p130Cas, pY416 Src, and anti-cleaved caspase-3 were from Cell Signaling Technology. Anti-pY397 FAK and TOPRO-3 were from Invitrogen. Anti-GAPDH (glyceraldehyde-3-phosphate dehydrogenase) was from Chemicon, bovine plasma fibronectin was from Sigma, and Dasatinib and PP1 were from LC Laboratories and Calbiochem, respectively. 4T1 murine mammary carcinoma cells and MDA-MB-231 human breast carcinoma cells were from American Type Culture Collection. ID8 mouse ovarian carcinoma cells were from Katherine Roby (Roby K F, Taylor C C, Sweetwood J P, Cheng Y, Pace J L, Tawfik O, et al. Development of a syngeneic mouse model for events related to ovarian cancer. Carcinogenesis 2000; 21:585-91). Cells were cultured in Dulbecco's modified Eagle's medium supplemented with 10% fetal bovine serum (FBS), 1 mM non-essential amino acids, 2 mM glutamine, 100 U/ml penicillin, and 100 μg/ml streptomycin. For in vitro studies, PND-1186 was dissolved in dimethyl sulfoxide (DMSO) and stored at −80° C. until time of use. Final experimental DMSO concentration was between 0.1% to 0.2%. The coding sequence for red fluorescent mCherry or dsRed proteins (Clontech) were subcloned into the lentiviral expression vector (pCDH-MSCl, System Biociences) and recombinant lentivirus was produced as described in Mitra S K, Lim S T, Chi A, Schlaepfer D D. Intrinsic focal adhesion kinase activity controls orthotopic breast carcinoma metastasis via the regulation of urokinase plasminogen activator expression in a syngeneic tumor model. Oncogene 2006; 25:4429-40. Transduced 4T1 or ID8 cells were enriched by fluorescence-activated cell sorting (FACSAria, Becton-Dickinson) to acquire stable population of cells. Selection of highly metastatic mCherry 4T1 cells was performed by isolation and expansion of cells from lung metastases. mCherry-4T1 cells were harvested and injected into the T4 mammary fat pad of 8-10 week female Balb/c mice. After 4 weeks the lungs were removed, dissociated into single cells using elastase and collagenase treatments, and then cultured with 60 μM of 6-thioguanine (Sigma) for 2 weeks to select for 4T1 cells. A population of mCherry-4T1 cells (4T1-L) was obtained by fluorescence-activated cell sorting (FACS), treated with ciprofloxacin (10 μg/ml), verified to be mycoplasma-negative via polymerase chain reaction (Stratagene), and re-verified to establish spontaneous lung metastatic colonies within 10 days after breast fat pad injection.

Anchorage-dependent, spheroid, and soft agar cell growth assays: Cells (2×10⁵) were plated per 35 mm well under adherent (tissue culture-treated) and non-adherent conditions (poly-HEMA-coated) in 6-well plates (Costar) in growth media. Between 24 and 168 h, all cells were collected, a single cell suspension was prepared by limited trypsin-EDTA treatment, and viable cells were enumerated by trypan blue staining and counting (ViCell XR, Beckman). For spheroid area determination, cells were imaged after 72 h in phase contrast using an Olympus IX51 microscope. Area was calculated using Image J software (version 1.43). For soft agar assays, 48-well plates were coated with a 1:4 mix of 2% agar (EM Science) in 0.2 ml growth media (bottom layer). 5×10⁴ cells were plated per well (in triplicate) in a mixture of 0.3% agar in 0.2 ml growth media (top layer). After agar solidification, 0.2 ml growth media was added containing DMSO or PND-1186 (final concentration for 0.6 ml). In separate experiments, PND-1186 was added after 4 days. After 10 days, colonies were imaged in phase contrast, enumerated by counting 9 fields (3 fields per well), and total area determined using Image J. For all analyses, experimental points were performed in triplicate and were experiments were repeated at least two times.

Immunoblotting: Protein extracts of cells were made using lysis buffer containing 1% Triton X-100, 1% sodium deoxycholate, and 0.1% SDS and were separated by 4-12% SDS-PAGE (sodium dodecyl sulfate polyacrylamide gel electrophoresis) and sequential immunoblotting performed as described in Mitra S K, Lim S T, Chi A, Schlaepfer D D. Intrinsic focal adhesion kinase activity controls orthotopic breast carcinoma metastasis via the regulation of urokinase plasminogen activator expression in a syngeneic tumor model. Oncogene 2006; 25:4429-40. Relative expression levels and phospho-specific antibody reactivity were measured by densitometry analyses of blots using Image J (version 1.42q). Inhibition of FAK and p130Cas tyrosine phosphorylation was quantified by calculating the ratio of pY397 FAK protein to total FAK. Similar analyses were performed for p130Cas using pY410 p130Cas and total p130Cas blot data.

Immunohistochemistry: For the detection of apoptosis, sections (7 μm) were analyzed using a terminal deoxynucleotidyl transferase dUTP nick end labeling (TUNEL) kit (Roche). For CD45 staining, sections (7 μm) were fixed in 4% paraformaldehyde, rinsed in PBS, and blocked with a solution of PBS containing 5% BSA, 1% goat serum and 0.1% Triton X. FITC-conjugated anti-CD45 antibodies (Invitrogen) at 1 μg/ml in 5% BSA and PBS were incubated for 2 hours. FITC-conjugated IgG2b isotype antibodies (Invitrogen) at the same concentration were used as a negative control. Cell nuclei were visualized by incubation with 1:25,000 dilution of Heochst 33342 (Invitrogen). Images were sequentially captured at 40× (UPLFL objective, 1.3 NA; Olympus) using a monochrome charge-coupled camera (ORCA ER; Hamamatsu), an inverted microscope (IX51; Olympus), and Slidebook software (v5.0, Intelligent Imaging). Images were pseudo-colored, overlaid, and merged using Photoshop CS3 (Adobe). Fluorescence quantitation was performed using Image J (v1.43).

Cell migration assays: Serum-stimulated chemotaxis using Millicell (12 mm diameter with 8 μm pores; Millipore) chambers were performed as described previously⁴⁹. Both sides of membrane were coated with fibronectin (10 μg/ml) and chemotaxis was stimulated by addition of 10% FBS to the lower chamber. Data points represent cell counts (9 fields) from three migration chambers from at least two independent experiments. For scratch-wound closure motility assays, cells were seeded onto fibronectin-coated (10 μg/ml) glass bottom 12 well plates (MatTek) and serum starved (0.5% FBS) for 16 h. Cells were wounded with a pipette tip, washed with phosphate-buffered salin (PBS), and replenished with 10% FBS media with or without FAK inhibitor (1 μM). Time-lapse series was obtained by acquiring images at 10 min intervals for up to 22 h, at 37° C. with humidity and CO₂ regulation using a 10× objective on an automated stage (Olympus IX81). Cell trajectories and distance traveled were measured by tracking nucleus position over time using Image J.

Cell growth and apoptosis assays: For cell growth analyses, adherent or suspended cells were treated with PND-1186 for the indicated times, collected as a single cell suspension by limited trypsin treatment, fixed with 70% ethanol, collected by centrifugation and washed with PBS. Cell pellets were resuspended in 300 p. 1 of PBS containing propidium iodide (PI) (10 μg/ml), DNAse-free RNAse (100 μg/ml, Qiagen), and then incubated at 37° C. with agitation for 1 h. Samples were analyzed by flow cytometry (FACSCalibur, Becton-Dickinson) and cell cycle analyses were performed by ModFit LT3.2 software (Verity software house). Hypodiploid DNA content as a measure of cell apoptosis was detected by PI staining as described ²⁹. For cell apoptosis analyses, adherent or suspended cells were treated with PND-1186 and collected as above, stained for phycoerythrin (PE)-conjugated annexin V binding and 7-amino-actinomycin (7-AAD) reactivity (BD Pharmingen), and analyzed within 1 h by flow cytometry. Quadrant gates were positioned based on cell autofluorescence (negative) staurosporine-treated (positive) controls. Apoptosis was calculated to be the percent of annexin V-positive cells. In the soft agar assays, apoptosis was quantified by visual inspection of at least 200 cells and was defined as the appearance of membrane blebbing or cell shrinkage. Apoptosis was also detected by appearance of cleaved caspase-3 antibody reactivity in protein lysates by immunoblotting.

IL-6 ELISA: Two million 4T1 cells were plated and allowed to spread for 4 h in 10% FBS after which time, DMSO (control) or the indicated concentration of PND-1186 was added. After 1 h, recombinant tumor necrosis factor-α (TNFα eBioScience) was added (10 ng/ml) and after 24 h, IL-6 levels in conditioned media were measured using anti-mouse IL-6 ELISA kit (eBioScience).

Detection of apoptosis in tumors: Fresh tumors were snap-frozen in Optimal Cutting Temperature (OCT) compound (Tissue Tek), thin sectioned (7 μM) using a cryomicrotome (Leica 3050S) and mounted onto glass slides. Sections were fixed with 3% paraformaldehyde, permeabilized in PBS containing 0.1% Triton for 3 min, and blocked with 8% goat serum in PBS for 60 min at RT. For activated caspase-3 detection, sections were incubated with cleaved caspase-3 antibody (1:200 diluted in 2% goat serum in PBS) for 18 h at 4° C., washed with PBS, and incubated with fluorescein isothiocyanate conjugated anti-rabbit and TOPRO-3 (blue) for DNA detection. Sections were imaged on a Nikon Eclipse C1 confocal microscope with a 1.4 NA 60× oil objective, with a 30 μm pinhole setting, and analyzed using EZ-C1 3.50 software (Nikon). Tumor apoptosis was also measured by terminal deoxynucleotidyl transferase dUTP nick end labeling (TUNEL) staining using the tetramethylrhodamine (TMR) kit as per the manufacturer's instructions (Roche). Bright field and fluorescent images of whole tumor sections were obtained using Zeiss M2-Bio Stereo microscope equipped with INFINITY1-3C: digital color camera and a 4× objective.

Mouse tumor studies: Six to eight week old female C57B16 and BALB/c mice were obtained from Harlan Laboratories (Indianapolis, Ind.) and housed in pathogen-free conditions, according to the guidelines of the Association for the Assessment and Accreditation for Laboratory Animal Care, International. All in vivo studies were carried out under an approved institutional experimental animal care and use protocol. Growing tumor cells were harvested by limited trypsinization, washed in PBS, and counted using a ViCell XR (Beckman) prior to injection. Cell viability as measured by trypan blue exclusion was >95%. For subcutaneous tumor growth, 1×10⁶ mCherry-labeled 4T1 cells in 100 μl PBS were injected into the hindflank of Balb/C mice. After 8 days, mice with equal volume tumors (as measured using vernier calipers and determined by length×width²/2) were grouped (n=8 per group) and PND-1186 solubilized in polyethylene glycol 400 (PEG400) in PBS (1:1) was injected (100 μl) subcutaneously in the neck region at 30 mg/kg or 100 mg/kg every 12 hours. Control animals received PEG400:PBS injections and at 13 days, tumors were imaged in situ using an Olympus OV100 Intravital Fluorescence Molecular Imaging System, tumors were excised and weighed, half was frozen in OCT, and half was solubilized in protein lysis buffer for FAK phosphorylation analyses. For ID8 ovarian carcinoma tumor growth, 0.8 mL of 1×10⁷ ID8 cells in PBS was intraperitoneal injected into C57B16 mice. After 11 days, 0.5 mg/mL PND-1186 dissolved in 5% sucrose in water was provided for drinking and control mice received 5% sucrose (n=8 per group). Administration continued ad libitum for 30 days after which mice were euthanized, ascites fluid collected, cells obtained by centrifugation (2000 rpm for 5 min), cell volume measured by pipet, and then solubilized in protein lysis buffer for immunoblotting analyses.

Pharmacokinetic (PK) evaluation of PND-1186: For intravenous (i.v.) injections, mice were given vehicle or 2 mg/kg PND-1186 in 10% DMSO, 10% Tween 80, and 80% water. For intraperitoneal (i.p.) injections, mice were given 30 or 100 mg/kg PND-1186 in 50% PEG400 in PBS. For oral (p.o.) administration, mice were given 150 mg/kg PND-1186 in water. Blood samples were collected via terminal heart puncture at 0.5, 1, 2, 4, 8, 12, 24 and 48 h for p.o. administration and 0.083, 0.25, 0.5, 1, 2, 4, 8, 12, 24 and 48 h for i.p. and i.v. administration. 3 mice per time point were used. For ad libitum administration, blood samples were collected after 7 days using five mice per group. Samples were collected in tubes containing 0.05 ml 0.5 M EDTA, centrifuged at 900×g for 15 min at room temperature, and the plasma collected. PND-1186 content was determined by high-performance liquid chromatography (HPLC) and mass spectroscopy analyses (see Supplemental Methods).

Pharmacodynamic (PD) evaluation of PND-1186: 4T1 cells were injected in the flank of Balb/c mice and allowed to grow as tumors (300-400 mm³) for 10 days. Vehicle (50% PEG400 in PBS), 30 or 100 mg/kg PND-1186 were i.p. injected and mice were sacrificed at 1, 3, 6 and 12 hours. Five mice were used per group. Tumors were resected and homogenized using a Pro 200 tissue homogenizer (Pro Scientific) in lysis buffer containing 1% Triton-X 100, 50 mM Hepes pH 7.4, 150 mM NaCl, 10% Glycerol, 1.5 mM MgCl₂, 1 mM EGTA, 10 mM sodium pyrophosphate, 100 mM NaF, 1 mM sodium orthovanadate, 10 μg/ml leupeptin, 10 μg/ml aprotinin. Protein concentration in lysates was determined using the micro bicinchoninic acid kit (Thermo). Equal protein lysates were resolved by SDS-PAGE and analyzed by immunoblotting.

Apoptosis assay: Suspended cells were treated with PND-1186, collected, stained for fluorescein-conjugated annexin V binding (30 min), and analyzed within 1 h by flow cytometry. Quadrant gates were positioned based on cell autofluorescence (negative) staurosporine-treated (positive) controls. Apoptosis was calculated to be the percent of annexin V-positive cells.

Orthotopic breast cancer models: One million 4T1 or MDA-MB-231 cells in 10 μl PBS were injected into the T4 mammary fat pad of 8-10 week old mice using a Hamilton syringe. PND-1186 treatment (oral gavage or ad libitum) was initiated when the tumors were palpable (24-48 hr for 4T1 and after 12 days for MDA-MB-231). Tumors were measured every 3-4 days with digital vernier calipers and tumor volume (mm³) was calculated using the formula: V=axb²/2 (a=length, mm; b=width, m) Body weight was measured weekly to assess toxicity. Lungs, spleen, and primary tumors were surgically removed and weighed. Tumors sections were homogenized in protein lysis buffer for immunoblotting or placed in Optimal Cutting Temperature (OCT) compound (Tissue Tek), frozen in liquid nitrogen, thin sectioned (7 μM) using a cryostat (Leica 3050S), and mounted onto glass slides.

For 4T1 tumor metastasis analyses, lungs were rinsed in PBS, dorsal and ventral fluorescent images acquired using the OV100 Small Animal Imaging System (Olympus). For all images, a common threshold for mCherry fluorescence was set and lung metastatic burden was calculated by determining the average integrated pixel density for micro-tumors present in each lung using Image J software. Metastatic tumor burden (number of metastatic lesion or mean pixel volume) was determined and groups (Negligible, Moderate, and High) were separated based upon numbers distribution. After imaging, lungs were fixed in Bouin's solution (Sigma), paraffin embedded, sectioned, and stained with hematoxylin and eosin (H&E) for histological evaluation. Images were acquired using a differential interference contrast-equipped Olympus IX81 inverted microscope and an Olympus DP71 digital color camera using Slidebook (v5.0) software. For MDA-MB-231 tumor metastasis studies, lungs were inflated by intratracheal injection of a 1:1 solution of OCT in sterile water using a 25 gauge needle. Lungs were resected, embedded in OCT, and frozen in liquid nitrogen. Average number of lung metastases per lobe was determined by enumerating lung lesions in H&E sections (n=11 lobes for sucrose and n=13 lobes for PND-1186).

Experimental Metastasis Assay: Twelve week old nude mice were administered 150 mg/kg PND-1186 or water (vehicle) p.o. at 14 and 2 hours prior to the i.v. (via tail vein) injection of 0.5 million (in 100 μl PBS) MDA-MB-231 cells stably-expressing mCherry fluorescent protein. To determine experimental metastasis burden, lungs were removed 1 and 6 h post cell injection, rinsed in PBS, and dorsal plus ventral fluorescent images acquired using OV100 imaging. A common threshold for mCherry fluorescence was set for all images and the total fluorescent lung area was calculated using Image J.

Statistical Methods: Significant difference between groups was determined using one-way ANOVA with Tukey post hoc. Differences between pairs of data were determined using an unpaired two-tailed student's t-test or a two-tailed Mann-Whitney test. Differences between metastasis incidences were determined using a two-tailed Fisher's exact test. All statistical analyses were performed using GraphPad Prism (version 5.0b, GraphPad Software, San Diego Calif.). p-values of <0.05 were considered significant.

### REFERENCES

1. Schlaepfer D D, Hauck C R, Sieg D J. Signaling through focal adhesion kinase. Prog Biophys Mol Biol 1999; 71:435-78.
2. Mitra S K, Hanson D A, Schlaepfer D D. Focal adhesion kinase: in command and control of cell motility. Nat Rev Mol Cell Biol 2005; 6:56-68.
3. Tomar A, Schlaepfer D D. Focal adhesion kinase: switching between GAPs and GEFs in the regulation of cell motility. Curr Opin Cell Biol 2009; 21:676-83.
4. McLean G W, Carragher N O, Avizienyte E, Evans J, Brunton V G, Frame M C. The role of focal-adhesion kinase in cancer—a new therapeutic opportunity. Nat Rev Cancer 2005; 5:505-15.
5. Zhao J, Guan J L. Signal transduction by focal adhesion kinase in cancer. Cancer Metastasis Rev 2009; 28:35-49.
6. Parsons J T, Slack-Davis J, Tilghman R, Roberts W G. Focal adhesion kinase: targeting adhesion signaling pathways for therapeutic intervention. Clin Cancer Res 2008; 14:627-32.
7. Benlimame N, He Q, Jie S, Xiao D, Xu Y J, Loignon M, et al. FAK signaling is critical for ErbB-2/ErbB-3 receptor cooperation for oncogenic transformation and invasion. J Cell Biol 2005; 171:505-16.
8. Luo M, Fan H, Nagy T, Wei H, Wang C, Liu S, et al. Mammary epithelial-specific ablation of the focal adhesion kinase suppresses mammary tumorigenesis by affecting mammary cancer stem/progenitor cells. Cancer Res 2009; 69:466-74.
9. Provenzano P P, Inman D R, Eliceiri K W, Beggs H E, Keely P J. Mammary epithelial-specific disruption of focal adhesion kinase retards tumor formation and metastasis in a transgenic mouse model of human breast cancer. Am J Pathol 2008; 173:1551-65.
10. Lahlou H, Sanguin-Gendreau V, Zuo D, Cardiff R D, McLean G W, Frame M C, et al. Mammary epithelial-specific disruption of the focal adhesion kinase blocks mammary tumor progression. Proc Natl Acad Sci USA 2007; 104:20302-7.
11. Pylayeva Y, Gillen K M, Gerald W, Beggs H E, Reichardt L F, Giancotti F G. Ras- and PI3K-dependent breast tumorigenesis in mice and humans requires focal adhesion kinase signaling. J Clin Invest 2009; 119:252-66.
12. van Nimwegen M J, Verkoeijen S, van Buren L, Burg D, van de Water B. Requirement for focal adhesion kinase in the early phase of mammary adenocarcinoma lung metastasis formation. Cancer Res 2005; 65:4698-706.
13. Mitra S K, Lim S T, Chi A, Schlaepfer D D. Intrinsic focal adhesion kinase activity controls orthotopic breast carcinoma metastasis via the regulation of urokinase plasminogen activator expression in a syngeneic tumor model. Oncogene 2006; 25:4429-40.
14. Mitra S K, Schlaepfer D D. Integrin-regulated FAK-Src signaling in normal and cancer cells. Curr Opin Cell Biol 2006; 18:516-23.
15. Shi Q, Hjelmeland A B, Keir S T, Song L, Wickman S, Jackson D, et al. A novel low-molecular weight inhibitor of focal adhesion kinase, TAE226, inhibits glioma growth. Mol Carcinog 2007; 46:488-96.
16. Slack-Davis J K, Martin K H, Tilghman R W, Iwanicki M, Ung E J, Autry C, et al. Cellular characterization of a novel focal adhesion kinase inhibitor. J Biol Chem 2007; 282:14845-52.
17. Roberts W G, Ung E, Whalen P, Cooper B, Hulford C, Autry C, et al. Antitumor activity and pharmacology of a selective focal adhesion kinase inhibitor, PF-562,271. Cancer Res 2008; 68:1935-44.
18. Golubovskaya V M, Nyberg C, Zheng M, Kweh F, Magis A, Ostrov D, et al. A small molecule inhibitor, 1,2,4,5-benzenetetraamine tetrahydrochloride, targeting the y397 site of focal adhesion kinase decreases tumor growth. J Med Chem 2008; 51:7405-16.
19. Liu T J, LaFortune T, Honda T, Ohmori O, Hatakeyama S, Meyer T, et al. Inhibition of both focal adhesion kinase and insulin-like growth factor-I receptor kinase suppresses glioma proliferation in vitro and in vivo. Mol Cancer Ther 2007; 6:1357-67.
20. Halder J, Lin Y G, Merritt W M, Spannuth W A, Nick A M, Honda T, et al. Therapeutic efficacy of a novel focal adhesion kinase inhibitor TAE226 in ovarian carcinoma. Cancer Res 2007; 67:10976-83.
21. Golubovskaya V M, Virnig C, Cance W G. TAE226-Induced apoptosis in breast cancer cells with overexpressed Src or EGFR. Mol Carcinog 2007; 47:222-34.
22. Hochwald S N, Nyberg C, Zheng M, Zheng D, Wood C, Massoll N A, et al. A novel small molecule inhibitor of FAK decreases growth of human pancreatic cancer. Cell Cycle 2009; 8:2435-43.

23. Lim S-T, Mikolon D, Stupack D G, Schlaepfer D D. FERM control of FAK function: Implications for cancer therapy. Cell Cycle 2008; 7:2306-14.
24. Weis S M, Lim S T, Lutu-Fuga K M, Barnes L A, Chen X L, Gothert J R, et al. Compensatory role for Pyk2 during angiogenesis in adult mice lacking endothelial cell FAK. J Cell Biol 2008; 181:43-50.
25. Zouq N K, Keeble J A, Lindsay J, Valentijn A J, Zhang L, Mills D, et al. FAK engages multiple pathways to maintain survival of fibroblasts and epithelia: differential roles for paxillin and p130Cas. J Cell Sci 2009; 122:357-67.
26. Wu L, Bernard-Trifilo J A, Lim Y, Lim S T, Mitra S K, Uryu S, et al. Distinct FAK-Src activation events promote alpha5beta1 and alpha4beta1 integrin-stimulated neuroblastoma cell motility. Oncogene 2008; 27:1439-48.
27. Defilippi P, Di Stefano P, Cabodi S. p130Cas: a versatile scaffold in signaling networks. Trends Cell Biol 2006; 16:257-63.
28. Sieg D J, Hauck C R, Ilic D, Klingbeil C K, Schaefer E, Damsky C H, et al. FAK integrates growth-factor and integrin signals to promote cell migration. Nat Cell Biol 2000; 2:249-56.
29. Nicoletti I, Migliorati G, Pagliacci M C, Grignani F, Riccardi C. A rapid and simple method for measuring thymocyte apoptosis by propidium iodide staining and flow cytometry. J Immunol Methods 1991; 139:271-9.
30. Mazumder S, Plesca D, Almasan A. Caspase-3 activation is a critical determinant of genotoxic stress-induced apoptosis. Methods Mol Biol 2008; 414:13-21.
31. Friedrich J, Seidel C, Ebner R, Kunz-Schughart L A. Spheroid-based drug screen: considerations and practical approach. Nat Protoc 2009; 4:309-24.
32. Almeida E A, Ilic D, Han Q, Hauck C R, Jin F, Kawakatsu H, et al. Matrix survival signaling: from fibronectin via focal adhesion kinase to c-Jun NH(2)-terminal kinase. J Cell Biol 2000; 149:741-54.
33. Shield K, Ackland M L, Ahmed N, Rice G E. Multicellular spheroids in ovarian cancer metastases: Biology and pathology. Gynecol Oncol 2009; 113:143-8.
34. Frisch S M, Vuori K, Ruoslahti E, Chan-Hui P Y. Control of adhesion-dependent cell survival by focal adhesion kinase. J Cell Biol 1996; 134:793-9.
35. Hungerford J E, Compton M T, Matter M L, Hoffstrom B G, Otey C A. Inhibition of pp125FAK in cultured fibroblasts results in apoptosis. J Cell Biol 1996; 135:1383-90.
36. Stupack D G, Cheresh D A. Get a ligand, get a life: integrins, signaling and cell survival. J Cell Sci 2002; 115:3729-38.
37. Lim S T, Miller N L, Nam J O, Chen X L, Lim Y, Schlaepfer D D. PYK2 inhibition of p53 as an adaptive and intrinsic mechanism facilitating cell proliferation and survival. J Biol Chem 2009; 285:1743-53.
38. Ilic D, Almeida E A, Schlaepfer D D, Dazin P, Aizawa S, Damsky C H. Extracellular matrix survival signals transduced by focal adhesion kinase suppress p53-mediated apoptosis. J Cell Biol 1998; 143:547-60.
39. Sawada Y, Tamada M, Dubin-Thaler B J, Chemiayskaya O, Sakai R, Tanaka S, et al. Force sensing by mechanical extension of the Src family kinase substrate p130Cas. Cell 2006; 127:1015-26.
40. Cho S Y, Klemke R L. Extracellular-regulated kinase activation and CAS/Crk coupling regulate cell migration and suppress apoptosis during invasion of the extracellular matrix. J Cell Biol 2000; 149:223-36.
41. Cabodi S, Tinnirello A, Di Stefano P, Bisaro B, Ambrosino E, Castellano I, et al. p130Cas as a new regulator of mammary epithelial cell proliferation, survival, and HER2-neu oncogene-dependent breast tumorigenesis. Cancer Res 2006; 66:4672-80.
42. Provenzano P P, Keely P J. The role of focal adhesion kinase in tumor initiation and progression. Cell Adh Migr 2009; 3:347-50.
43. Michael K E, Dumbauld D W, Burns K L, Hanks S K, Garcia A J. FAK Modulates Cell Adhesion Strengthening via Integrin Activation. Mol Biol Cell 2009; 20:2508-19.
44. Frisch S M. Caspase-8: fly or die. Cancer Res 2008; 68:4491-3.
45. Walsh C, Tanjoni I, Uryu S, Nam J O, Mielgo A, Tomar A, et al. Oral delivery of PND-1186 FAK inhibitor decreases spontaneous breast to lung metastasis in pre-clinical tumor models. Cancer Biology & Therapy 2010; (submitted).
46. Wu Y M, Tang J, Zhao P, Chen Z N, Jiang J L. Morphological changes and molecular expressions of hepatocellular carcinoma cells in three-dimensional culture model. Exp Mol Pathol 2009; 87:133-40.
47. Liang C, Koenig M, He Y, Holmberg P Inhibitors of Focal Adhesion Kinase. World Intellectual Property Organization 2008: WO2008115369.
48. Roby K F, Taylor C C, Sweetwood J P, Cheng Y, Pace J L, Tawfik O, et al. Development of a syngeneic mouse model for events related to ovarian cancer. Carcinogenesis 2000; 21:585-91.
49. Lim Y, Lim S T, Tomar A, Gardel M, Bernard-Trifilo J A, Chen X L, et al. PyK2 and FAK connections to p190Rho guanine nucleotide exchange factor regulate RhoA activity, focal adhesion formation, and cell motility. J Cell Biol 2008; 180:187-203.

While the invention has been described and exemplified in sufficient detail for those skilled in this art to make and use it, various alternatives, modifications, and improvements will be apparent to those skilled in the art without departing from the spirit and scope of the claims.

All patents and publications referred to herein are incorporated by reference herein to the same extent as if each individual publication was specifically and individually indicated to be incorporated by reference in its entirety.

The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1. A method of promoting apoptosis in tumor cells of a patient, or inhibiting metastasis in a patient, or both, comprising administering an effective amount of an inhibitor of focal adhesion kinase to the patient in need thereof, wherein the inhibitor is a compound of formula 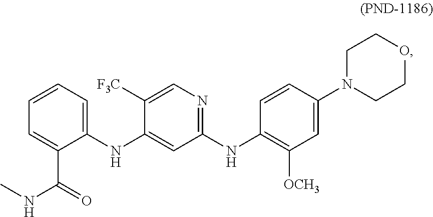
or any pharmaceutically acceptable salt thereof.

2. The method of claim 1 wherein promoting apoptosis results in inhibiting tumor growth, inhibiting tumor metastasis, or promoting tumor apoptosis, or any combination thereof, in the patient afflicted with a tumor.

3. The method of claim 2 wherein the tumor is a malignant cancer.

4. The method of claim 2 wherein the tumor comprises breast cancer or ovarian cancer.

5. The method of claim 1 wherein the inhibitor is administered to the patient in a formulation comprising a pharmaceutically acceptable excipient.

6. The method of claim 1 wherein the inhibitor is administered to the patient orally.

7. The method of claim 1 wherein the inhibitor is administered to the patient parenterally.

8. The method of claim 1 comprising multiple administrations of the inhibitor to the patient over a period of time for a duration and at a frequency sufficient to provide a beneficial effect to the patient.

9. The method of claim 1 further comprising administering an effective amount of a second medicament to the patient.

10. Use of an inhibitor of a Focal Adhesion Kinase inhibitor for preparation of a medicament for promoting apoptosis in tumor cells, or inhibiting metastasis in a patient, or both, wherein the inhibitor comprises a compound of formula 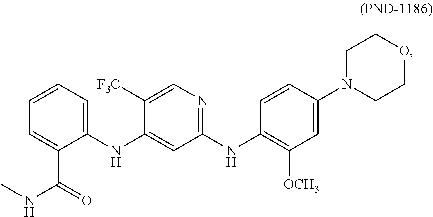
or any pharmaceutically acceptable salt thereof.

11. The use of claim 10 wherein promoting apoptosis results in inhibiting tumor growth, or inhibiting tumor metastasis, or promoting tumor apoptosis, or any combination thereof.
